(19) United States
(12) Patent Application Publication
(10) Pub. No.: US 2026/0178969 A1
(43) Pub. Date: Jun. 25, 2026

(54) MET SCORE: A CLINICAL DECISION SUPPORT SYSTEM

(71) Applicants: UNIVERSITY OF HOUSTON SYSTEM, Houston, TX (US); BAYLOR COLLEGE OF MEDICINE, Houston, TX (US)

(72) Inventors: Ioannis A. Kakadiaris, Houston, TX (US); Abraham Bautista-Castillo, Houston, TX (US); Tiphanie Vogel, Houston, TX (US)

(73) Assignees: UNIVERSITY OF HOUSTON SYSTEM, Houston, TX (US); BAYLOR COLLEGE OF MEDICINE, Houston, TX (US)

(21) Appl. No.: 19/124,860

(22) PCT Filed: Oct. 25, 2023
(86) PCT No.: PCT/US2023/035925

§ 371 Date: Apr. 26, 2025

Related U.S. Application Data
(60) Provisional application No. 63/419,460, filed on Oct. 26, 2022.

(51) Int. Cl. G06N 20/00; G16H 10/60; G16H 50/20

(52) U.S. Cl. CPC G06N 20/00; G16H 10/60; G16H 50/20

(57) ABSTRACT

Embodiments of the present disclosure pertain to methods and clinical decision support systems for distinguishing between a first and a second condition in a subject. Additional embodiments of the present disclosure pertain to computer-implemented methods of distinguishing between the first and the second condition in a subject. Further embodiments of the present disclosure pertain to computing devices that are operable to distinguish between the first and the second condition in a subject.

### CROSS-REFERENCE TO RELATED APPLICATIONS

This application claims priority to U.S. Provisional Patent Application No. 63/419,460, filed on Oct. 26, 2022. The entirety of the aforementioned application is incorporated herein by reference.

### STATEMENT REGARDING FEDERALLY SPONSORED RESEARCH

This invention was made with government support under R61 HD105593, awarded by the National Institutes of Health. The government has certain rights in the invention.

### BACKGROUND

Complexities exist in distinguishing between various conditions, such as inflammation and infection. Numerous embodiments of the present disclosure aim to address the aforementioned complexities.

### SUMMARY

In some embodiments, the present disclosure pertains to methods of distinguishing between a first and a second condition in a subject. In some embodiments, the methods of the present disclosure include: (1) receiving a first set of health-related data from the subject; (2) determining if a confidence index crosses a certain threshold; and (3a) distinguishing between the first and the second condition in the subject from the first set of health-related data if the confidence index crosses the threshold, or (3b) receiving a second set of health-related data from the subject if the confidence index does not cross the threshold and distinguishing between the first and the second condition in the subject from the first set and the second set of health-related data. In some embodiments, the methods of the present disclosure also include a step of making a treatment decision based on distinguishing between the first and the second condition in the subject.

Additional embodiments of the present disclosure pertain to clinical decision support systems for distinguishing between a first and a second condition in a subject. The clinical decision support systems of the present disclosure generally include: (1) instructions for receiving a first set of health-related data from the subject; (2) instructions for determining if a confidence index crosses a certain threshold; and (3a) instructions for distinguishing between the first and the second condition in the subject from the first set of health-related data if the confidence index crosses the threshold, or (3b) instructions for receiving a second set of health-related data from the subject if the confidence index does not cross the threshold and distinguishing between the first and the second condition in the subject from the first set and the second set of health-related data. In some embodiments, the systems of the present disclosure also include instructions for making a treatment decision based on distinguishing between the first and the second condition in the subject.

Additional embodiments of the present disclosure pertain to computer-implemented methods of distinguishing between a first and a second condition in a subject. In some embodiments, the computer-implemented methods of the present disclosure include: (1) receiving a plurality of health-related data from the subject, where the receiving includes feeding the health-related data to a machine-learning algorithm trained on the plurality of health-related data; and (2) distinguishing between the first and the second condition in the subject from the plurality of health-related data, where the distinguishing includes generating an output from the machine-learning algorithm.

Further embodiments of the present disclosure pertain to computing devices that are operable to distinguish between a first and a second condition in a subject. In some embodiments, the computing devices of the present disclosure include: (1) a machine-learning algorithm trained on a plurality of health-related data; (2) programming instructions for receiving a plurality of health-related data from the subject and feeding the health-related data into the machine-learning algorithm; and (3) programming instructions for generating an output from the machine-learning algorithm that distinguishes between the first and the second condition in the subject from the plurality of health-related data.

### DESCRIPTION OF THE DRAWINGS

FIG. 1A provides a method of distinguishing between a first and a second condition in a subject.

FIG. 1B provides a computing device for distinguishing between a first and a second condition in a subject.

FIG. 2 provides a flowchart of a two-stage clinical decision support system for distinguishing between multisystem inflammatory syndrome in children (MIS-C) from endemic typhus (AI-MET).

FIG. 3 illustrates a process where a feature is converted to categorical inputs by using set thresholds.

FIG. 4 illustrates a process where missing laboratory values are converted into a zero-value vector, enabling them to be handled by a deep learning model.

FIG. 5 illustrates a process occurring after the categorization process, where the inputs are related to those obtained from the same feature due to the possibility of all inputs being zero, generating contextual information.

FIG. 6 provides a visualization of the attention long short-term memory (LSTM) architecture used as part of a deep learning model (MET-30).

FIG. 7 illustrates a recurrent dropout applied to an LSTM cell in the LSTM layer. The dashed arrow signals the dropout applied to the part that updates the state of the cell.

FIGS. 8A-8B illustrate a standard dropout applied to the dense layers in MET-30.

FIG. 9 illustrates confidence index functions for MET-17.

FIGS. 10A-10C provide a summary of testing and training datasets.

FIG. 11 provides a summary of average attention values obtained from the MET-30 attention module. The attention values obtained from every experiment were scaled using the min-max scaling method. After that, the average attention values were computed.

FIG. 12 provides a summary of the machine learning model performance for experiment 1 in Example 1.

FIG. 13 summarizes the machine learning models performance for experiment 2 in Example 1.

FIG. 14 provides a percentage of patients from each dataset who were classified in each AI-MET stage.

FIGS. 15A-15B provides timelines for MIS-C and typhus patients. FIG. 15A shows that, in total, 87 typhus patients admitted from Jan. 1, 2020 to Dec. 31, 2021 were included in the training cohort. FIG. 15B shows that, in total, 293 MIS-C patients were included. Solid bars depict the 133 MIS-C patients in the training cohort from the original (May to November 2020) MIS-C surge and the delta variant surge (September to October 2021). Unfilled bars represent the 160 MIS-C patients considered for the validation cohort. Circles denote zero patients were admitted during that month.

FIGS. 16A-I summarize the MET-30 demographic features and vital signs from the training cohort's 133 MIS-C and 87 typhus patients. FIG. 16A shows that typhus patients were a median of 12 years of age and MIS-C patients nine years of age. FIG. 16B shows that 49% of typhus patients and 58% of MIS-C patients self-reported male sex. FIG. 16C shows that 17% of typhus patients and 68% of MIS-C patients reported a close exposure to someone with COVID-19 preceding their presentation. FIG. 16D shows that 7% of typhus and 38% of MIS-C patients reported an antecedent illness preceding their presentation. FIG. 16E shows that typhus patients had a median of 7 days of fever before presentation, and MIS-C patients had a median of 5 days. FIG. 16F shows the median maximum temperature reported on a presentation for typhus patients was 39.4 Celsius (° C.) and for MIS-C patients it was 39.7° C. FIG. 16G shows the median lowest systolic blood pressure (SBP) within 2 hours of presentation for typhus patients was 108 millimeters of mercury (mmHg) and 96 mmHg for MIS-C patients. FIG. 16H shows the median lowest diastolic blood pressure (DBP) within 2 hours of presentation for typhus patients was 58 mmHg, and it was 53 mmHg for MIS-C patients. FIG. 16I shows the median highest heart rate (HR) within 2 hours of presentation for typhus patients was 113 beats per minute (bpm), and it was 135 bpm for MIS-C patients. Red lines depict median values. Mann-Whitney U test was used for continuous variables, and Fisher's exact test for categorical variables. **** p<0.0001.

FIGS. 17A-J summarizes the MET-30 clinical features reported or observed in the training cohort's 133 MIS-C and 87 typhus patients. FIG. 17A shows that conjunctivitis was present in 39% of typhus patients and 72% of MIS-C patients. FIG. 17B shows that oromucosal changes were present in 15% of typhus patients and 28% of MIS-C patients. FIG. 17C shows hand or foot edema or erythema was not present in any typhus patient (black dot) but was found in 10% of MIS-C patients. FIG. 17D shows that rash was present in 80% of typhus patients and 51% of MIS-C patients. FIG. 17E shows t cervical adenopathy was present in 14% of typhus patients and 7% of MIS-C patients. FIG. 17F shows that gastrointestinal (GI) symptoms were present in 75% of typhus patients and 92% of MIS-C patients. FIG. 17G shows that musculoskeletal (MSK) symptoms were present in 59% of typhus patients and 37% of MIS-C patients. FIG. 17H shows neurologic (neuro) symptoms, including headache, were present in 67% of typhus patients and 56% of MIS-C patients. FIG. 17I shows that respiratory symptoms were present in 38% of typhus patients and 35% of MIS-C patients. FIG. 17J shows that cardiac symptoms were present in 9% of typhus patients and 11% of MIS-C patients. Fisher's exact test was used for categorical variables. **** p<0.0001, *** p<0.001, ** p<0.01, * p<0.05.

FIGS. 18A-18L summarize the MET-30 laboratory results at presentation in the training cohort's 133 MIS-C and 87 typhus patients. FIG. 18A shows that the median white blood cell count (WBC) was 7-3 K/μL in typhus patients and 9-4 K/μL in MIS-C patients. FIG. 18B shows that the median absolute neutrophil count (ANC) was 4-7 K/μL in typhus patients and 7-4 K/μL in MIS-C patients. FIG. 18C shows the median absolute lymphocyte count (ALC) was 1.9 K/μL in typhus patients and 0-96 K/μL in MIS-C patients. FIG. 18D shows that the median neutrophil to lymphocyte (NLR) ratio was 2-8 in typhus patients and 7 in MIS-C patients. Note the log axis. FIG. 18E shows that the median platelet value was 161 K/μL in typhus patients and 155 K/μL in MIS-C patients. FIG. 18F shows that the median sodium (Na) was 135 mmol/L in typhus patients and 133 mmol/L in MIS-C patients. FIG. 18G shows that the median aspartate aminotransferase (AST) was 121 U/L in typhus patients and 52 U/L in MIS-C patients. FIG. 18H shows that the median alanine aminotransferase (ALT) was 80 U/L in typhus patients and 37 U/L in MIS-C patients. FIG. 18I shows that the median lactate dehydrogenase (LDH) was 925 U/L in typhus patients and 367 U/L in MIS-C patients. FIG. 18J shows that the median fibrinogen was 394 mg/dL in typhus patients and 563 mg/dL in MIS-C patients. FIG. 18K shows that the median troponin I was normal (0-01 ng/mL) in typhus patients and 0-05 ng/mL in MIS-C patients. The y-axis is first broken at the upper limit of normal, 0-03 ng/mL. FIG. 18L shows that the median B-type natriuretic peptide (BNP) was 12 μg/mL in typhus patients and 167 μg/mL in MIS-C patients. The y-axis is first broken at the upper limit of normal, 200 μg/mL. Red lines depict median values. Mann-Whitney U test was used for continuous variables. **** p<0.0001; K, thousand.

FIG. 19 shows the two phases of AI-MET. A child presenting with a febrile illness may be suspected of having typhus or MIS-C, and AI-MET can be applied to distinguish between these. In Phase 1, 17 features are used to apply points from a fixed scoring system to classify patients as typhus or MIS-C without utilizing a computer system or electronic device. However, if the MET-17 score does not total sufficient points to cross the established confidence index, classifying the patient requires Phase 2 using MET-30. For MET-30, 13 features (30) are added, and a recurrent neural network classifies the patient as having typhus or MIS-C.

### DETAILED DESCRIPTION

It is to be understood that both the foregoing general description and the following detailed description are illustrative and explanatory and are not restrictive of the subject matter, as claimed. In this application, the use of the singular includes the plural, the word “a” or “an” means “at least one,” and the use of “or” means “and/or” unless specifically stated otherwise. Furthermore, the use of the term “including,” as well as other forms, such as “includes” and “included”, is not limiting. Also, terms such as “element” or “component” encompass elements or components comprising one unit and elements or components including more than one unit unless specifically stated otherwise.

The section headings used herein are for organizational purposes and are not construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials define a term in a manner that contradicts the definition of that term in this application, this application controls.

Complexities exist in distinguishing between various conditions, such as inflammation and infection. For instance, symptoms of typhus can mimic Multisystem Inflammatory Syndrome in Children (MIS-C) symptoms, such as the presence of fever, headache, rash, conjunctivitis, gastrointestinal complaints (e.g., pain, vomiting, diarrhea), and myalgias. Severe presentations of inflammation and infection can include life-threatening effects requiring intensive care, such as myocarditis, endocarditis, rhabdomyolysis, and multiorgan dysfunction.

Such complexities were observed in 2019 and 2020 during the Coronavirus disease 2019 (COVID-19) pandemic. In December 2019, the first Coronavirus disease 2019 (COVID-19) cases were reported, and several clinical challenges subsequently appeared. At that time, even though it was recognized that children were as susceptible to COVID-19 as adults, this infection was less frequently observed in pediatric patients than in adults (1-5% of all COVID-19 cases) and tended to present with a milder clinical course.

However, in April 2020, some children began to be hospitalized at an increased rate due to fever and multisystem inflammation. In May 2020, the Centers for Disease Control and Prevention (CDC) published a case definition for MIS-C. Some of the most frequent features found in MIS-C patients are fever, rash, conjunctivitis, oromucosal changes, abdominal pain, vomiting, diarrhea, myocarditis, and hematologic abnormalities. These findings overlap other diseases such as Kawasaki Disease (KD), toxic shock syndrome (TSS), and murine typhus, provoking a clinical challenge to distinguish MIS-C from these pathologies.

Clinical Decision Support Systems (CDSS) are computer systems designed to support the decision-making of medical teams about their patients and intended to improve care with clinical knowledge and patient information. These systems are often divided into knowledge and non-knowledge systems, where knowledge systems are those that make use of the information supplied by medical personnel to create the rules they use. In contrast, non-knowledge systems use artificial intelligence, machine learning, or statistical methods in lieu of instructions obtained from medical personnel. Advances in these fields have significantly increased the presence of CDSS in the medical field to address clinical challenges such as antibiotic management, heart disease prediction, and cancer detection.

With its clinical and laboratory similarities to MIS-C, distinguishing typhus has become a diagnostic challenge in the US where it is endemic: Texas, southern California, and Hawaii. Timing has become crucial, as early identification and targeted therapy are desirable for better outcomes in both conditions. This opens the opportunity for implementing a CDSS to help the Emergency Department (ED) medical staff make timely decisions.

CDSS has been used during the COVID-19 pandemic as support tools for the prognosis of disease severity for predicting mortality. However, only a few have focused on MIS-C-related clinical challenges. For instance, studies have used Random Forest as a model to achieve a high-sensitivity MIS-C diagnosis and to provide insights about the importance of features such as procalcitonin, ferritin, N-terminal pro-B-type natriuretic peptide (NT-proBNP), and C-reactive protein (CRP). A point to consider for CDSS is how missing values are treated, with the median value based on the presence or absence of MIS-C in this approach.

On the other hand, other studies proposed using logistic regression with bootstrap backward selection to identify the most relevant predictors for MIS-C. This model identified patients with MIS-C among an otherwise undifferentiated group of febrile patients with features obtained within the first 24 hours of hospital presentation.

Additional studies built a two-stage model of feed-forward neural networks. These models differentiate between MIS-C, KD, and children with nonspecific febrile illnesses. In the first stage of this model, the patient can be classified as MIS-C, not MIS-C, or rejected by the conformal prediction framework. If the patient is classified as not MIS-C, data will pass to the next stage and be used to differentiate between a febrile child and a KD patient. For the conformal prediction framework, they used trust sets by filtering patients in the training cohort with more than one missing value and an MIS-C risk score more significant than the 95^{th} percentile.

Even though these CDSS models focused on the clinical challenge that MIS-C represents, none considered endemic typhus as one of the possible overlapping febrile diseases of childhood, and none presented an alternative tool to be implemented in the ED without using electronic devices.

Therefore, a need exists for accelerating the diagnosis of various conditions (e.g., inflammation or infection) in a patient, especially during the first hours of a patient's visit to a hospital or clinic, which can in turn help result in fewer complications and better administration of clinical resources. A need also exists for a CDSS capable of distinguishing between patients with inflammation (e.g., MIS-C) or infection (e.g., endemic typhus) during the first hours of their presentation and implementation without needing an electronic device. Numerous embodiments of the present disclosure aim to address the aforementioned needs.

### Methods and Systems for Distinguishing Between Conditions

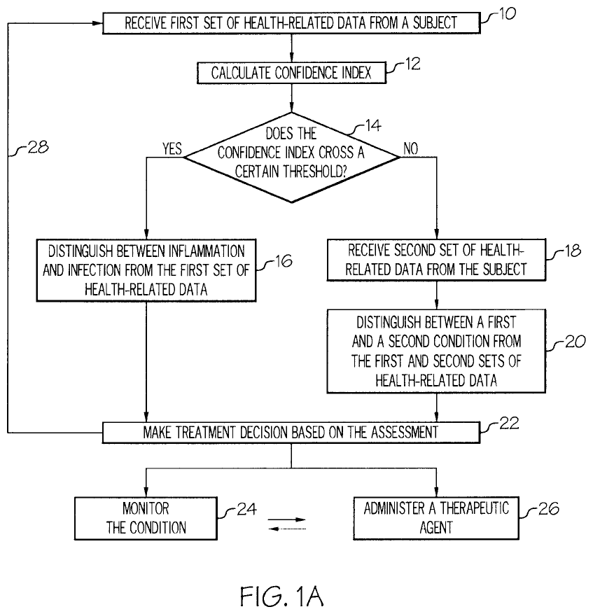

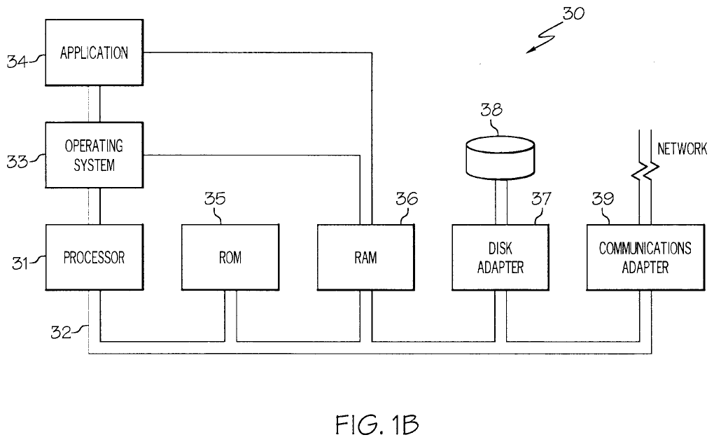

In some embodiments, the present disclosure pertains to methods of distinguishing between a first and a second condition in a subject. In some embodiments illustrated in FIG. 1A, the methods of the present disclosure include: receiving a first set of health-related data from the subject (step 10); determining if a confidence index crosses a certain threshold (14); and distinguishing between the first and the second condition in the subject from the first set of health-related data if the confidence index crosses the threshold (step 16), or receiving a second set of health-related data from the subject (step 18) if the confidence index does not cross the threshold and distinguishing between the first and the second condition in the subject from the first set and the second set of health-related data (step 20). In some embodiments, the methods of the present disclosure also include a step of calculating a confidence index from the first set of health-related data (step 12).

In some embodiments, the methods of the present disclosure also include a step of making a treatment decision based on distinguishing between the first and the second condition in the subject (step 22). For instance, the treatment decision can include monitoring the condition (step 24) and/or administering a therapeutic agent to the subject (step 26). In some embodiments, the methods of the present disclosure may be repeated after implementing the treatment decision (step 28).

Additional embodiments of the present disclosure pertain to clinical decision support systems for distinguishing between a first and a second condition in a subject. The clinical decision support systems of the present disclosure generally include: instructions for receiving a first set of health-related data from the subject; instructions for determining if a confidence index crosses a certain threshold; and instructions for distinguishing between the first and the second condition in the subject from the first set of health-related data if the confidence index crosses the threshold, or instructions for receiving a second set of health-related data from the subject if the confidence index does not cross the threshold and distinguishing between the first and the second condition in the subject from the first set and the second set of health-related data. In some embodiments, the systems of the present disclosure also include instructions for calculating a confidence index from the first set of health-related data.

In some embodiments, the systems of the present disclosure also include instructions for making a treatment decision based on distinguishing between the first and the second condition in the subject. In some embodiments, the treatment decision includes monitoring the course of the condition, administering a therapeutic agent to the subject, and combinations thereof.

As set forth in more detail herein, the methods and systems of the present disclosure can have numerous embodiments.

### Health-Related Data

The methods and systems of the present disclosure may utilize various types of health-related data. For instance, in some embodiments, the first set and the second set of health-related data each independently include, without limitation, demographic data, clinical data, vital signs, laboratory test results, and combinations thereof.

In some embodiments, at least one of the first set and the second set of health-related data include laboratory test results. In some embodiments, the laboratory test results include, without limitation, white blood cell (WBC) count, absolute lymphocyte count (ALC), absolute neutrophil count (ANC), platelet count, sodium count, troponin count, B-type natriuretic peptide (BNP) count, fibrinogen count, D-dimer count, aspartate aminotransaminase count (AST), lactate dehydrogenase count (LDH), alanine aminotransaminase count (ALT), ANC/ALC ratio, ferritin count, and combinations thereof.

In some embodiments, at least one of the first set and the second set of health-related data includes clinical data. In some embodiments, the clinical data include, without limitation, antecedent illness; conjunctivitis; epidemiologic link to COVID-19; oromucosal changes; hand or foot edema or erythema; rash; cervical adenopathy; nausea, vomiting, pain, diarrhea, or other gastrointestinal involvements; arthralgias, myalgias, or other musculoskeletal involvements; altered mental status (AMS), seizure, paresthesias, headache, or other neurological involvements; shortness of breath, cough, congestion, or other respiratory involvements; chest pain, palpitations or other cardiac involvements; and combinations thereof.

In some embodiments, at least one of the first set and the second set of health-related data include vital signs. In some embodiments, the vital signs include, without limitation, fever, heart rate, blood pressure, and combinations thereof.

In some embodiments, at least one of the first set and the second set of health-related data include demographic data. In some embodiments, the demographic data include, without limitation, age, gender (e.g., biological sex), and combinations thereof.

In some embodiments, the first set and the second set of health-related data each independently include, without limitation, age; gender (e.g., biological sex); epidemiologic link to COVID-19; antecedent illness; fever; maximum reported or recorded temperature during the first six hours of the subject's visit to a clinic or hospital; lowest systolic blood pressure during the first six hours of the subject's visit to a clinic or hospital; lowest diastolic blood pressure during the first six hours of the subject's visit to a clinic or hospital; highest heart rate during the first six hours of the subject's visit to a clinic or hospital; conjunctivitis; oromucosal changes; hand or foot edema or erythema; rash; cervical adenopathy; nausea, vomiting, pain, diarrhea, or other gastrointestinal involvements; arthralgias, myalgias, or other musculoskeletal involvements; altered mental status (AMS), seizure, paresthesias, headache, or other neurological involvements; shortness of breath, cough, congestion, or other respiratory involvements; chest pain, palpitations or other cardiac involvements; white blood cell (WBC) count; absolute neutrophil count (ANC); absolute lymphocyte count (ALC); platelet count; sodium count; aspartate aminotransferase (AST) count, alanine aminotransferase (ALT) count; lactate dehydrogenase (LDH) count; fibrinogen count; troponin count; B-type natriuretic peptide (BNP) count; ANC/ALC ratio; and combinations thereof.

Health-related data may be obtained at various points in time. For instance, in some embodiments, the first set and the second set of health-related data are obtained during the first six hours of the subject's visit to a clinic or hospital.

The health-related data may have various data points. For instance, in some embodiments, the first set of health-related data includes at least 10 data points. In some embodiments, the first set of health-related data includes at least 17 data points. In some embodiments, the second set of health-related data includes at least 10 data points. In some embodiments, the second set of health-related data includes at least 13 data points. In some embodiments, the combined first and second sets of health-related data include at least 30 data points.

### Confidence Indexes

The methods and systems of the present disclosure may consider various confidence indexes from the first set of health-related data. For instance, in some embodiments, the confidence index is a scoring system. In some embodiments, two thresholds are established for a scoring system in the form of scores. If the subject obtains a score equal to or higher than the upper threshold, it is established that the subject suffers from a first condition (e.g., an infection, such as endemic typhus). If the subject has a score equal to or lower than the lower threshold, it is found that the subject suffers from a second condition (e.g., inflammation, such as MIS-C). If the subject obtains a score within the range of both thresholds, it is considered that the confidence index has not been achieved, and therefore, the next stage of the system must be used.

Confidence indexes may have various thresholds. For instance, in some embodiments, the threshold includes a threshold established for a reliable diagnosis of a condition (e.g., inflammation or infection). In some embodiments, a confidence index is established based on the distribution of the values obtained from the scoring system for subjects affected by one or more conditions (e.g., inflammation and infection). In some embodiments, a single threshold is established for each condition.

### Distinguishing Between a First and a Second Condition

The methods and systems of the present disclosure may distinguish between a first and a second condition in a subject in various manners. For instance, in some embodiments, the distinguishing includes a diagnosis of the first or second condition (e.g., diagnosis of infection or inflammation).

In some embodiments, the methods and systems of the present disclosure occur manually. In some embodiments, the methods and systems of the present disclosure occur through the utilization of a computing device.

In some embodiments, at least the steps or instructions for receiving the first set of health-related data from the subject; and determining if the confidence index crosses a certain threshold occur without the use of a computing device. In some embodiments, at least the steps or instructions for receiving the second set of health-related data from the subject if the confidence index does not cross the threshold; and distinguishing between the first and the second condition in the subject from the first set and the second set of health-related data occurs through the utilization of a computing device.

### Machine-Learning Algorithms

In some embodiments, the computing device includes a machine-learning algorithm trained on the first and second sets of health-related data. In some embodiments, the receiving of the first set and second set of health-related data includes feeding the health-related data to the machine-learning algorithm. In some embodiments, the distinguishing between a first and a second condition includes generating an output from the machine-learning algorithm.

The methods and systems of the present disclosure may utilize various types of machine-learning algorithms. For instance, in some embodiments, the machine-learning algorithm is an Li-regularized logistic regression algorithm. In some embodiments, the machine learning algorithm includes supervised learning algorithms. In some embodiments, the supervised learning algorithms include nearest neighbor algorithms, naïve-Bayes algorithms, decision tree algorithms, linear regression algorithms, support vector machines, neural networks, convolutional neural networks, ensembles (e.g., random forests and gradient-boosted decision trees), and combinations thereof.

In some embodiments, the machine learning algorithm includes: an attention module operable to receive health-related data as inputs and provide values that weigh the inputs based on relevance; a long short-term memory (LSTM) layer operable to receive the weighted inputs from the attention module and learn contextual information within the inputs; and a classification module operable to receive the inputs from the LSTM layer, where the classification module includes a plurality of connected dense layers, and where the dense layers are operable to distinguish between a first and a second condition and generate a corresponding output. In some embodiments, the attention module includes at least one dense layer. In some embodiments, the LSTM layer includes a plurality of LSTM cells. In some embodiments, the output of the LSTM layer feeds a set of dense layers that function as the classification module. In some embodiments, this module can be replaced by any other classification module that uses another artificial intelligence algorithm such as support vector machine, Xboost, decision tree, logistic regression, and the like.

The machine learning algorithms of the present disclosure may be trained in various manners. For instance, in some embodiments, the training includes: (1) feeding health-related data into a machine learning algorithm, where the health-related data are from one or more subjects that have developed one or more conditions (e.g., inflammation and/or infection); (2) feeding another set of health-related data into the machine learning algorithm, where the health-related data are from one or more subjects that have not developed the one or more conditions (e.g., an infection and/or inflammation); and (3) training the machine learning algorithm to distinguish between the conditions (e.g., infection and inflammation) by comparing the aforementioned categories of health-related data. In some embodiments, training a machine learning algorithm includes adjusting weights or parameters within the machine learning algorithm to differentiate between the aforementioned categories of health-related data. In some embodiments, training a machine learning algorithm includes providing health-related data relevance values to differentiate between the aforementioned categories.

In some embodiments where the machine learning algorithm includes an attention module, an LSTM layer, and a classification module, training includes feeding health-related data through the attention module capable of providing values that weigh the inputs based on relevance. Thereafter, the outputs of the attention module are multiplied by the inputs to focus on the relevance learned by the attention module to facilitate learning the deep model. The result of this multiplication feeds an LSTM layer whose function is to understand the contextual information of the features. The LSTM layer's outputs can then provide fully connected layers that perform the subject classification. In some embodiments, the patients' features go through an attention module capable of providing values that weigh the inputs based on relevance. The outputs of the attention module are multiplied by the inputs to focus on the relevance learned by the attention module to facilitate learning the deep model. The result of this multiplication feeds an LSTM layer whose function is to understand the contextual information of the features. Its outputs provide fully connected layers that perform the patient classification.

### Conditions

The methods and systems of the present disclosure may be utilized to distinguish between various types of first and second conditions. For instance, in some embodiments, each of the first condition and the second condition independently includes inflammation or infection. In some embodiments, the first condition includes inflammation and the second condition includes infection. In some embodiments, each of the first condition and the second condition includes inflammation. In some embodiments, each of the first and second conditions independently includes, without limitation, Multisystem Inflammatory Syndrome in Children (MIS-C), Kawasaki Disease, a febrile illness, typhus, endemic typhus, or toxic shock syndrome (TSS). In some embodiments, the inflammation includes Multisystem Inflammatory Syndrome in Children (MIS-C). In some embodiments, the inflammation includes Kawasaki Disease. In some embodiments, the inflammation includes a febrile illness. In some embodiments, the infection includes endemic typhus. In some embodiments, the infection includes toxic shock syndrome (TSS). In some embodiments, the methods and systems of the present disclosure may be utilized to distinguish between MIS-C (i.e., a first condition) and endemic typhus (i.e., a second condition). In some embodiments, the methods and systems of the present disclosure may be utilized to distinguish between MIS-C (i.e., a first condition) and TSS (i.e., a second condition). In some embodiments, the methods and systems of the present disclosure may be utilized to distinguish between MIS-C (i.e., a first condition) and a febrile illness (i.e., a second condition).

### Subjects

The methods and systems of the present disclosure may be utilized to distinguish between a first and a second condition in various subjects. For instance, in some embodiments, the subject is a human being. In some embodiments, the subject is a human being under the age of 18. In some embodiments, the subject suffers from a condition (e.g., inflammation or infection).

### Measuring Health-Related Data

In some embodiments, the methods and systems of the present disclosure also include a step of, or instructions for, measuring or obtaining the first set and second set of health-related data. For instance, in some embodiments, the first set and the second set of health-related data are measured or obtained from a tissue sample, a body fluid, or a blood sample of the subject. In some embodiments, the methods and systems of the present disclosure also include a step of, or instructions for, obtaining a tissue sample, body fluid, or blood sample from the subject and measuring the first set and the second set of health-related data from the tissue sample, body fluid or blood sample obtained from the subject.

### Treatment Decisions

In some embodiments, the methods and systems of the present disclosure also include a step of, or instructions for, making a treatment decision based on distinguishing between a first and a second condition in the subject. For instance, in some embodiments, the treatment decision includes monitoring the course of the condition, administering a therapeutic agent to the subject, and combinations thereof. In some embodiments, the treatment decision includes monitoring the course of the condition. In some embodiments, the treatment decision includes administering a therapeutic agent to the subject. In some embodiments, the therapeutic agent includes, without limitation, anti-viral drugs, anti-inflammatories, steroids, antibiotics, and combinations thereof. In some embodiments, the method is repeated after implementing the treatment decision.

### Computing Devices and Methods for Distinguishing Between Conditions

Additional embodiments of the present disclosure pertain to computer-implemented methods of distinguishing between a first and a second condition in a subject. In some embodiments, the computer-implemented methods of the present disclosure include: (1) receiving a plurality of health-related data from the subject, where the receiving includes feeding the health-related data to a machine-learning algorithm trained on the plurality of health-related data; and (2) distinguishing between a first and a second condition in the subject from the plurality of health-related data, where the distinguishing includes generating an output from the machine-learning algorithm.

Further embodiments of the present disclosure pertain to computing devices that are operable to distinguish between a first and a second condition in a subject. In some embodiments, the computing devices of the present disclosure include: (1) a machine-learning algorithm trained on a plurality of health-related data; (2) programming instructions for receiving a plurality of health-related data from the subject and feeding the health-related data into the machine-learning algorithm; and (3) programming instructions for generating an output from the machine-learning algorithm that distinguishes between a first and a second condition in the subject from the plurality of health-related data. As set forth in more detail herein, the computer-implemented methods and computing devices of the present disclosure can have numerous embodiments.

### Health-Related Data

The computer-implemented methods and computing devices of the present disclosure can receive and utilize various health-related data. Suitable health-related data were described supra and are incorporated herein by reference. For instance, in some embodiments, the health-related data include, without limitation, demographic data, clinical data, vital signs, laboratory test results, and combinations thereof.

In some embodiments, the health-related data include laboratory test results. In some embodiments, the laboratory test results include, without limitation, white blood cell (WBC) count, absolute lymphocyte count (ALC), absolute neutrophil count (ANC), platelet count, sodium count, troponin count, B-type natriuretic peptide (BNP) count, fibrinogen count, D-dimer count, aspartate aminotransaminase count (AST), lactate dehydrogenase count (LDH), alanine aminotransaminase count (ALT), ANC/ALC ratio, ferritin count, and combinations thereof.

In some embodiments, the health-related data include clinical data. In some embodiments, the clinical data include, without limitation, antecedent illness; conjunctivitis; epidemiologic link to COVID-19; oromucosal changes; hand or foot edema or erythema; rash; cervical adenopathy; nausea, vomiting, pain, diarrhea, or other gastrointestinal involvements; arthralgias, myalgias, or other musculoskeletal involvements; altered mental status (AMS), seizure, paresthesias, headache, or other neurological involvements; shortness of breath, cough, congestion, or other respiratory involvements; chest pain, palpitations or other cardiac involvements; and combinations thereof.

In some embodiments, the health-related data include vital signs. In some embodiments, the vital signs include, without limitation, fever, heart rate, blood pressure, and combinations thereof.

In some embodiments, the health-related data include demographic data. In some embodiments, the demographic data include, without limitation, age, gender (e.g., biological sex), and combinations thereof.

In some embodiments, the health-related data include, without limitation, age; gender (e.g., biological sex); epidemiologic link to COVID-19; antecedent illness; fever; maximum reported or recorded temperature during the first six hours of the subject's visit to a clinic or hospital; lowest systolic blood pressure during the first six hours of the subject's visit to a clinic or hospital; lowest diastolic blood pressure during the first six hours of the subject's visit to a clinic or hospital; highest heart rate during the first six hours of the subject's visit to a clinic or hospital; conjunctivitis; oromucosal changes; hand or foot edema or erythema; rash; cervical adenopathy; nausea, vomiting, pain, diarrhea, or other gastrointestinal involvements; arthralgias, myalgias, or other musculoskeletal involvements; altered mental status (AMS), seizure, paresthesias, headache, or other neurological involvements; shortness of breath, cough, congestion, or other respiratory involvements; chest pain, palpitations or other cardiac involvements; white blood cell (WBC) count; absolute neutrophil count (ANC); absolute lymphocyte count (ALC); platelet count; sodium count; aspartate aminotransferase (AST) count, alanine aminotransferase (ALT) count; lactate dehydrogenase (LDH) count; fibrinogen count; troponin count; B-type natriuretic peptide (BNP) count; ANC/ALC ratio; and combinations thereof.

Health-related data may be obtained at various time points. For instance, in some embodiments, the health-related data are obtained during the first six hours of the subject's visit to a clinic or hospital. In some embodiments, the health-related data include at least 10 data points. In some embodiments, the health-related data include at least 30 data points. In some embodiments, the health-related data include at least 17 data points. In some embodiments, the health-related data include at least 30 data points.

### Machine-Learning Algorithms

The computer-implemented methods and computing devices of the present disclosure can utilize various types of machine-learning algorithms. Suitable machine-learning algorithms were described supra and are incorporated herein by reference.

In some embodiments, the machine learning algorithm includes a supervised machine learning algorithm. In some embodiments, the machine learning algorithm includes: an attention module, where the attention module is operable to receive health-related data as inputs and provide values that weigh the inputs based on relevance; a long short-term memory (LSTM) layer operable to receive the weighted inputs from the attention module and learn contextual information within the inputs; and a classification module operable to receive the inputs from the LSTM layer, where the classification module includes a plurality of connected dense layers, and where the dense layers are operable to distinguish between a first and a second condition and generate a corresponding output. In some embodiments, the attention module includes at least one dense layer. In some embodiments, the LSTM layer includes a plurality of LSTM cells.

### Distinguishing Between Conditions

The computer-implemented methods and computing devices of the present disclosure can be utilized to distinguish between a first and a second condition in various manners. For instance, in some embodiments, distinguishing between a first and a second condition in the subject includes a diagnosis of the condition.

The computer-implemented methods and computing devices of the present disclosure can be utilized to distinguish between various first and second conditions. For instance, in some embodiments, each of the first condition and the second condition independently includes inflammation or infection. In some embodiments, the first condition includes inflammation and the second condition includes infection. In some embodiments, each of the first condition and the second condition includes inflammation. In some embodiments, each of the first and second conditions independently includes, without limitation, Multisystem Inflammatory Syndrome in Children (MIS-C), Kawasaki Disease, a febrile illness, typhus, endemic typhus, or toxic shock syndrome (TSS). In some embodiments, the inflammation includes Multisystem Inflammatory Syndrome in Children (MIS-C). In some embodiments, the inflammation includes Kawasaki Disease. In some embodiments, the inflammation includes a febrile illness. In some embodiments, the infection includes endemic typhus. In some embodiments, the infection includes toxic shock syndrome (TSS). In some embodiments, the computer-implemented methods and computing devices of the present disclosure may be utilized to distinguish between MIS-C (i.e., a first condition) and endemic typhus (i.e., a second condition). In some embodiments, the computer-implemented methods and computing devices of the present disclosure may be utilized to distinguish between MIS-C (i.e., a first condition) and TSS (i.e., a second condition). In some embodiments, the computer-implemented methods and computing devices of the present disclosure may be utilized to distinguish between MIS-C (i.e., a first condition) and a febrile illness (i.e., a second condition).

The computer-implemented methods and computing devices of the present disclosure can be utilized to distinguish between a first and a second condition in various subjects. For instance, in some embodiments, the subject is a human being. In some embodiments, the subject is a human being under the age of 18. In some embodiments, the subject suffers from a condition (e.g., inflammation or infection).

### Treatment Decisions

In some embodiments, the computer-implemented methods of the present disclosure also include a step of making a treatment decision based on distinguishing between a first and a second condition in the subject. In some embodiments, the computing devices of the present disclosure also include programming instructions for making a treatment decision based on distinguishing between a first and a second condition in the subject.

In some embodiments, the treatment decision includes monitoring the course of the condition, administering a therapeutic agent to the subject, and combinations thereof. In some embodiments, the treatment decision includes monitoring the course of the condition. In some embodiments, the treatment decision includes administering a therapeutic agent to the subject. In some embodiments, the therapeutic agent includes, without limitation, anti-viral drugs, anti-inflammatories, steroids, antibiotics, and combinations thereof.

### Computing Devices

Computing devices used in the present disclosure can have numerous variations. For instance, the computing devices of the present disclosure can include various types of computer-readable storage mediums. In some embodiments, the computer-readable storage mediums can be a tangible device that can retain and store instructions for use by an instruction execution device. In some embodiments, the computer-readable storage medium may include, without limitation, an electronic storage device, a magnetic storage device, an optical storage device, an electromagnetic storage device, a semiconductor storage device, and combinations thereof. A non-exhaustive list of more specific examples of suitable computer-readable storage medium includes, without limitation, a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, and combinations thereof.

A computer-readable storage medium, as used herein, is not to be construed as being transitory signals per se. Such transitory signals may be represented by radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer-readable program instructions described herein can be downloaded to respective computing/processing devices from a computer-readable storage medium or to an external computer or external storage device via a network, such as the Internet, a local area network, a wide area network and/or a wireless network. In some embodiments, the network may include copper transmission cables, optical transmission fibers, wireless transmission, routers, firewalls, switches, gateway computers and/or edge servers. In some embodiments, a network adapter card or network interface in each computing/processing device receives computer-readable program instructions from the network and forwards the computer-readable program instructions for storage in a computer-readable storage medium within the respective computing/processing device.

In some embodiments, computer-readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object-oriented programming language such as Smalltalk, C++, or the like, and procedural programming languages, such as the “C” programming language or similar programming languages.

In some embodiments, the computer-readable program instructions may execute entirely on the user's computer as a stand-alone software package, partly on the user's computer and partly on a remote computer or entirely on the remote computer or server. In the latter scenario, the remote computer may be connected in some embodiments to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry in order to perform aspects of the present disclosure.

Embodiments of the present disclosure as discussed herein may be implemented using a computing device illustrated in FIG. 1B. Referring now to FIG. 1B, FIG. 1B illustrates an embodiment of the present disclosure of the hardware configuration of a computing device 30 represents a hardware environment for practicing various embodiments of the present disclosure.

Computing device 30 has a processor 31 connected to various other components by system bus 32. An operating system 33 runs on processor 31 and provides control and coordinates the functions of the various components of FIG. 1B. An application 34 in accordance with the principles of the present disclosure runs in conjunction with operating system 33 and provides calls to operating system 33, where the calls implement the various functions or services to be performed by application 34. Application 34 may include, for example, a program for distinguishing between a first and a second condition as discussed in the present disclosure, such as in connection with FIGS. 1A, 2-14, 15A-15B, 16A-16I, 17A-17J, 18A-18L, and 19.

Referring again to FIG. 1B, read-only memory (“ROM”) 35 is connected to system bus 32 and includes a basic input/output system (“BIOS”) that controls certain basic functions of computing device 30. Random access memory (“RAM”) 36 and disk adapter 37 are also connected to system bus 32. It should be noted that software components including operating system 33 and application 34 may be loaded into RAM 36, which may be computing device's 30 main memory for execution. Disk adapter 37 may be an integrated drive electronics (“IDE”) adapter that communicates with a disk unit 38 (e.g., a disk drive). It is noted that the program for distinguishing between a first and a second condition, as discussed in the present disclosure, such as in connection with FIGS. 1A, 2-14, 15A-15B, 16A-16I, 17A-17J, 18A-18L, and 19, may reside in disk unit 38 or in application 34.

Computing device 30 may further include a communications adapter 39 connected to bus 32. Communications adapter 39 interconnects bus 32 with an outside network (e.g., wide area network) to communicate with other devices.

Aspects of the present disclosure are described herein with reference to flowchart illustrations and/or block diagrams of methods, apparatus (systems), and computing devices according to embodiments of the disclosure. It will be understood that computer-readable program instructions can implement each block of the flowchart illustrations and/or block diagrams, and combinations of blocks in the flowchart illustrations and/or block diagrams.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart and/or block diagram block or blocks. The computer-readable program instructions may also be loaded onto a computer, other programmable data processing apparatus, or other devices to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computing devices according to various embodiments of the present disclosure. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter.

### Example 1. AI-MET: a Deep Learning-Based Clinical Decision Support System for Distinguishing Multisystem Inflammatory Syndrome in Children from Endemic Typhus

The COVID-19 pandemic brought several diagnostic challenges, including the post-infectious sequelae multisystem inflammatory syndrome in children (MIS-C). Some of the clinical features of this syndrome can be found in other pathologies such as Kawasaki disease, toxic shock syndrome, and endemic typhus. Endemic typhus, or murine typhus, is an acute infection treated much differently than MIS-C. As such, early detection is crucial to a favorable prognosis for patients with these disorders. Clinical Decision Support Systems (CDSS) are computer systems designed to support the decision-making of medical teams about their patients and intended to improve uprising clinical challenges in healthcare.

In this Example, Applicants present a CDSS to distinguish between MIS-C and typhus that includes a scoring system that allows the timely distinction of both pathologies only using clinical and laboratory features typically available within the first six hours of presentation to the Emergency Department. The proposed approach was trained and tested on datasets of 87 typhus patients and 133 MIS-C patients. A comparison was made against five well-known statistical and machine-learning models. A second dataset with 111 MIS-C patients was used to verify the AI-MET effectiveness and robustness. The performance assessment for AI-MET and the five statistical and machine learning models was performed by computing sensitivity, specificity, accuracy, and precision. The AI-MET system scores 100 percent in the five metrics used on the training and testing dataset and 99 percent on the validation dataset.

The main contributions of this Example are: (1) developing a two-stage CDSS for distinguishing between MIS-C and typhus; (2) creating a scoring system capable of being implemented in the ED without an electronic device; (3) optimizing the number of clinical features required to differentiate MIS-C from typhus; and (4) proposing a categorization process based on established thresholds for managing missing clinical values for Deep Learning architectures.

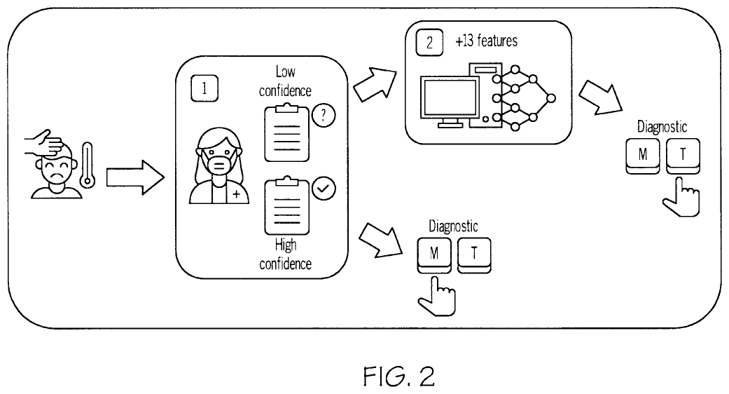

As illustrated in FIG. 2, Artificial Intelligence to distinguish MIS-C from endemic typhus, or AI-MET, is a decision support system divided into two main stages. During the first stage, MET-17, a scoring system, provides patients' classification and a confidence index for this classification without using a computer system or electronic device using only 17 features. If the classification doesn't meet the established confidence index, the medical team can appeal to the next stage, MET-30, where another 13 features are asked for a total of 30 features. MET-30 is a recurrent neural network with an attention module capable of distinguishing patients with MIS-C from patients with typhus and computing the importance of its inputs for this classification. Both stages receive their name due to the number of features needed to compute a possible diagnosis.

### Example 1.1. Pre-Processing

The preprocessing performed on the dataset includes the categorization and normalization of patient features. For the feature categorization process, Applicants used predefined thresholds that convert a feature into up to three inputs depending on the thresholds used (FIG. 3).

Laboratory features such as white blood cells (WBC), absolute lymphocyte count (ALC), absolute neutrophil count (ANC), platelet count, sodium, troponin, B-type natriuretic peptide (BNP), and fibrinogen were categorized as low, normal, and high using the thresholds established in Table 1.

TABLE 1
Established thresholds to categorize lab test results.

| Laboratory feature | Low | High |
|---|---|---|
| ALC (K/μL) | <1500 | n/a |
| ANC (K/μL) | n/a | >8000 |
| BNP (pg/mL) | n/a | >200 |
| D-dimer (mg/L) | n/a | >0.40 |
| Fibrinogen (mg/L) | <220 | >440 |
| NT-proBNP (pg/mL) | n/a | >125 |
| Platelets (×1000/μL) | <200 | >500 |
| Sodium (mmol/L) | <135 | n/a |
| Troponin (ng/ml) | n/a | >0.03 |
| WBC (K/μL) | <4 | >11 |

In cases such as aspartate aminotransaminase (AST), lactate dehydrogenase (LDH), and alanine aminotransaminase (ALT), Applicants used Tables 2, 3, and 4, which take into account the age and sex to define low, normal, and high.

TABLE 2
Established thresholds to categorize AST (U/L) lab tests.

| Age | 0 to | 4 to | 7 to | 10 to | 12 to | 14 to | 16 to |
|---|---|---|---|---|---|---|---|
| (years) | <4 | <7 | <10 | <12 | <14 | <16 | <21 |

Female

| | | | | | | | |
|---|---|---|---|---|---|---|---|
| Min | 20 | 15 | 15 | 10 | 10 | 10 | 5 |
| Max | 60 | 50 | 40 | 40 | 30 | 30 | 30 |

Male

| | | | | | | | |
|---|---|---|---|---|---|---|---|
| Min | 20 | 15 | 15 | 10 | 15 | 15 | 10 |
| Max | 60 | 50 | 40 | 60 | 40 | 40 | 45 |

TABLE 3
Established thresholds to categorize LDH (U/L) lab tests.

| | | | | | | | |
|---|---|---|---|---|---|---|---|
| Age | 0 to | 4 to | 7 to | 10 to | 12 to | 14 to | 16 |
| (years) | <4 | <7 | <10 | <12 | <14 | <16 | to <21 |

Female

| | | | | | | | |
|---|---|---|---|---|---|---|---|
| Min | 223 | 209 | 187 | 169 | 169 | 174 | 151 |
| Max | 409 | 401 | 334 | 343 | 285 | 258 | 298 |

Male

| | | | | | | | |
|---|---|---|---|---|---|---|---|
| Min | 223 | 209 | 187 | 192 | 209 | 160 | 151 |
| Max | 409 | 401 | 334 | 312 | 334 | 325 | 298 |

TABLE 4
Established thresholds to categorize ALT (U/L) lab tests.

| Age | 0 to | 4 to | 7 to | 10 to | 12 to | 14 to | 16 to |
|---|---|---|---|---|---|---|---|
| (years) | <4 | <7 | <10 | <12 | <14 | <16 | <21 |

Female

| | | | | | | | |
|---|---|---|---|---|---|---|---|
| Min | 14 | 11 | 11 | 11 | 11 | 10 | 10 |
| Max | 45 | 28 | 28 | 28 | 28 | 35 | 35 |

Male

| | | | | | | | |
|---|---|---|---|---|---|---|---|
| Min | 12 | 10 | 10 | 10 | 10 | 11 | 11 |
| Max | 45 | 41 | 41 | 41 | 41 | 26 | 26 |

All established thresholds were obtained from the Texas Children's Hospital (TCH) Pathology Catalog standard references. Demographics and clinical features were normalized using the function “normalize” from scikitlearn package preprocessing using l²-norm. Applicants established “1” as presence and “0” as absence for categorical features. Demographic, clinical, and laboratory features provided 73 inputs that were reduced to 40 (see Example 1.4) for the models shown in this Example.

### Example 1.2. Missing Values

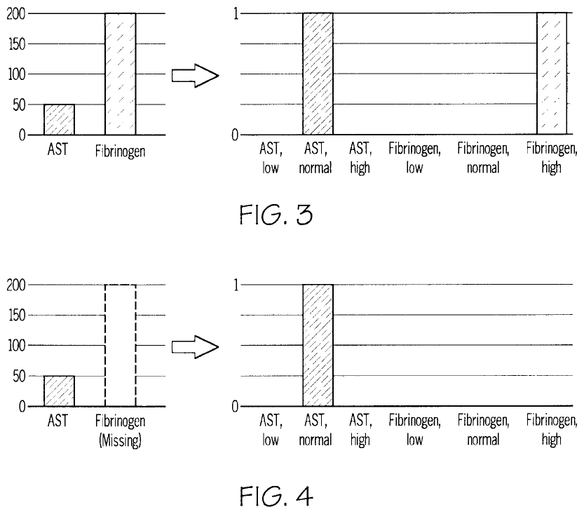

Missing values in datasets is a recurring problem in the healthcare area due to systematic (different data capture protocols used at other times) or non-deterministic (individuals or institutions not providing data) differences. As illustrated in FIG. 4, the categorization process based on established thresholds allows handling these missing laboratory values by turning them into zero-value inputs (FIG. 4). These features were converted into inputs with only zeros, describing the clinical features as neither high, normal, nor low, which enabled them to be handled by the deep learning model without the need to use any data imputation method for laboratory features that could create a bias during the learning process.

### Example 1.3. Contextual Information

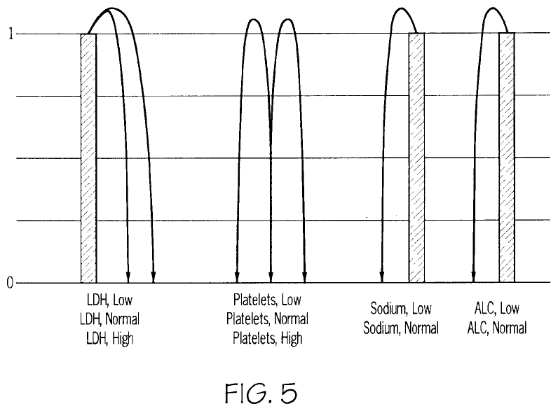

The different possible combinations of inputs generated from a feature (low, normal, high, or missing) provided contextual information from patient data generated by converting laboratory features into categorical inputs. That is, the significance of the patient input and its meaning depended not only on its value but on the value of other directly related inputs (inputs generated from the same laboratory feature) (FIG. 5). This design decision was essential when selecting the deep learning model architecture.

### Example 1.4. Feature Selection

The 40 out of 73 inputs were selected through an iterative process based on the values obtained by the attention module of Applicants' deep learning model (MET-30). During the model training, the inputs with the lowest values obtained from the attention module were eliminated one by one while ensuring that the testing accuracy of the model was not reduced. Once the testing accuracy was affected by the elimination of the input with the lowest value obtained, the iterative process of input selection was stopped, resulting in 40 inputs related to 30 features.

### Example 1.5. MET-17

To create MET-17, a scoring system that can be used in the ED without an electronic device, Applicants took the attention values of the inputs obtained from the MET-30 attention module and grouped them arbitrarily into four groups. Later, Applicants defined the score, r, as the formula shown in Equation 1.

$$τ = η + ω \qquad ( Equation 1 )$$

In Equation 1, η represents the categorical input values, and W the continuous input values. To compute the categorical input values, η, Applicants first defined an auxiliary variable φ in accordance with the formula shown in Equation 2.

$$ϕ = {\sum }_{m = 1}^{4} {\sum }_{n = 1}^{N} {G}_{m} * [ P ( T \vert {I}_{m}^{n} ) - P ( M \vert {I}_{m}^{n} ) ] \qquad ( Equation 2 )$$

In Equation 2, an attention value Gm was established for each group by taking the arithmetic mean of the attention values of all the inputs in the group. For each categorical input, I n, m, where
m denotes the group to which that input belongs, and n is the input index for that group, the group attention value was multiplied by the difference in the probability of that patient being classified as typhus given that input, (P (T|ln m)), minus the probability of that patient of being classified as MIS-C given the same input (P (M|ln m)). The values obtained from the auxiliary variable were normalized, multiplied by ten, and rounded, obtaining η as follows in Equation 3.

$$η = round ( 10 * [ \frac{ϕ}{‖ ϕ ‖} ] ) \qquad ( Equation 3 )$$

In Equation 3, all values below one were excluded. In the case of the continuous values, Applicants defined w under Equation 4.

$$ω = {\sum }_{m = 1}^{4} {\sum }_{j = 1}^{J} {K}_{m} * sign ( {T}_{m}^{j} - {C}_{m}^{j} ) \qquad ( Equation 4 )$$

In Equation 4, sign(x) will be one for x≥0 and zero otherwise, Tjm is an empirically established threshold set based on the distribution of the value of the input in question for patients with MIS-C and patients with typhus, Cjm is the patient continuous value where m denotes the attention group and f the input in that group, and Km is a constant determined based on which attention value group the input belonged to and the hierarchy K1>K2>K3>K4.

### Example 1.6. MET-30

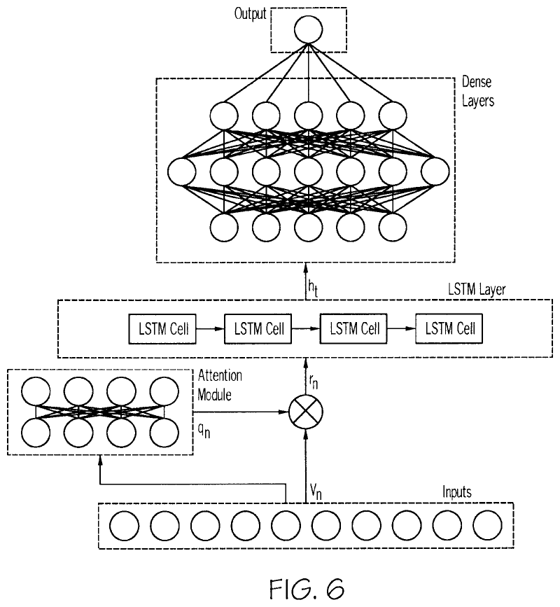

In contrast, MET-30 is a Deep Learning model consisting of three components: an attention module capable of providing values that weigh the inputs based on relevance, a long short-term memory (LSTM) layer that can learn the contextual information among the data, and a module of classification composed of dense layers (FIG. 6). The attention module of the model weighs the input values. Vn, by their importance for the correct classification of patients. This is done by a dense layer, un, that uses tanh as its activation function to maximize the weights of the inputs that contribute the most and minimize the weights of those with the smallest contribution and later computes the attention values among the inputs using a softmax function in the next layer, resulting in qn, which is multiplied by the inputs shown in Equation 5.

$${u}_{n} = f ( {V}_{n} ) = tanh ( {W}_{a} * {V}_{n} + {b}_{a} ) , \qquad ( Equation 5 )$$

$${q}_{n} = softmax ( {u}_{n} ) , {r}_{n} = {q}_{n} {V}_{n} .$$

The LSTM layer is constituted by LSTM cells defined by it; ft, and of that represent the input, forget, and output gates, while ct is the cell state and ht the hidden state as defined in Equation 6.

$${i}_{\text{?}} = σ ( {W}_{\text{?}} * [ {h}_{\text{?} - 1} , {r}_{n} ] + {b}_{\text{?}} ) , \qquad ( Equation 6 )$$

$${f}_{\text{?}} = σ ( {W}_{f} * [ {h}_{\text{?} - 1} , {r}_{n} ] + {b}_{f} ) ,$$

$${o}_{\text{?}} = σ ( {W}_{o} * [ {h}_{\text{?} - 1} , {r}_{n} ] + {b}_{o} ) ,$$

$${c}_{\text{?}} = {f}_{\text{?}} * {c}_{\text{?} - 1} + {i}_{\text{?}} * tanh ( {W}_{c} * [ {h}_{\text{?} - 1} , {r}_{n} ] + {b}_{c} ) ,$$

$${h}_{\text{?}} = {o}_{\text{?}} * tanh ( {c}_{\text{?}} ) .$$

$$\text{?} \text{indicates text missing or illegible when filed}$$

Equation 6 will be the input for the classification module formed by a set of densely connected layers.

### Example 1.7. Overfitting

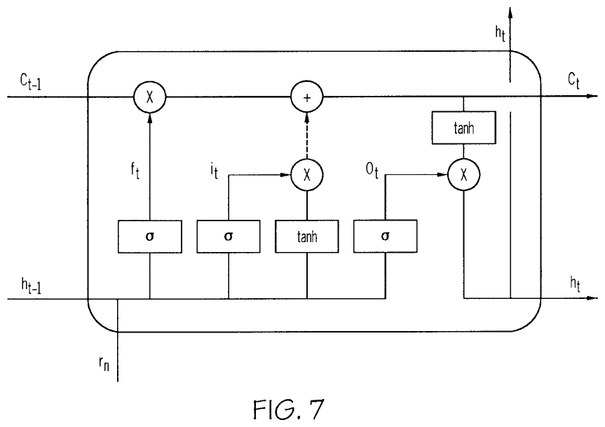

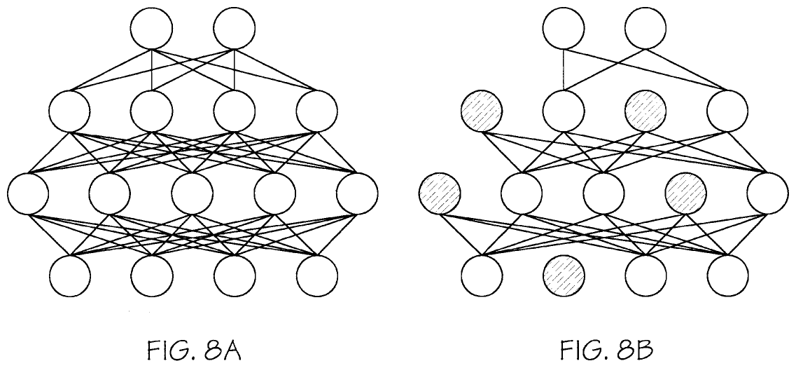

Architectures containing LSTM layers are prone to overfitting. This phenomenon occurs when a machine learning or deep learning model overfits its parameters during its training to “learn” in detail the features present in the training dataset, thus losing the ability to generalize the information and performing poorly on “unseen” data. To avoid this, a recurrent dropout was implemented in the LSTM layer (FIG. 7) with standard dropout (FIGS. 8A-8B) in subsequent dense layers of the architecture.

### Example 1.8. AI-MET: A Two-Stage Clinical Decision Support System

AI-MET is designed as a two-stage decision approach. In the first stage. patient information regarding the 17 features used by MET-17 is entered to classify the patient as typhus or MIS-C. If the score obtained, r, is insufficient to issue a reliable diagnosis, 13 more features are requested to be used in MET-30, the deep learning model.

To quantify the confidence of the score obtained by MET-17, Applicants designed a Confidence Index (CI) for patients with MIS-C, Hr, and for patients with endemic typhus, Tr, as follows in Equation 7.

$$M ( τ ) = - α + \frac{β}{exp ( - δ * τ + γ )} + 1 , \qquad ( Equation 7 )$$

$$T ( τ ) = α + \frac{β}{exp ( - δ * τ + γ )} ,$$

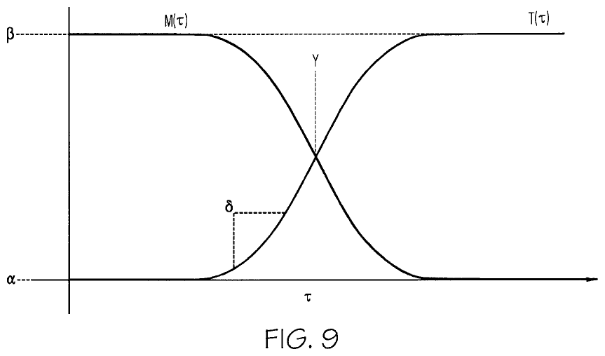

In Equation 7, α represents the bias, β the coefficient's maximum value, δ the function's growth rate, and γ as the cross-point between both functions for the distribution of the values obtained from MET-17 (FIG. 9).

### Example 1.9. Training/Testing Dataset Description

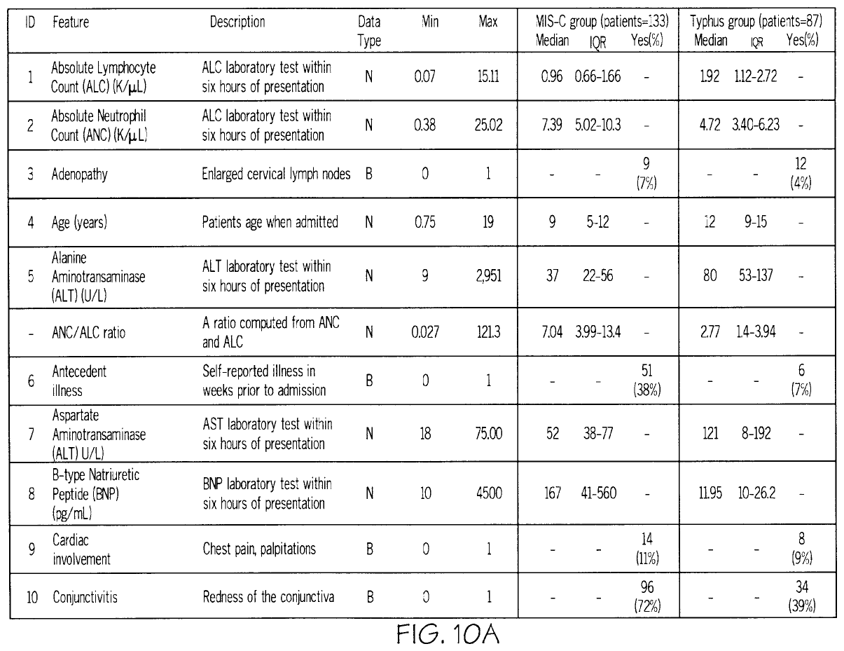

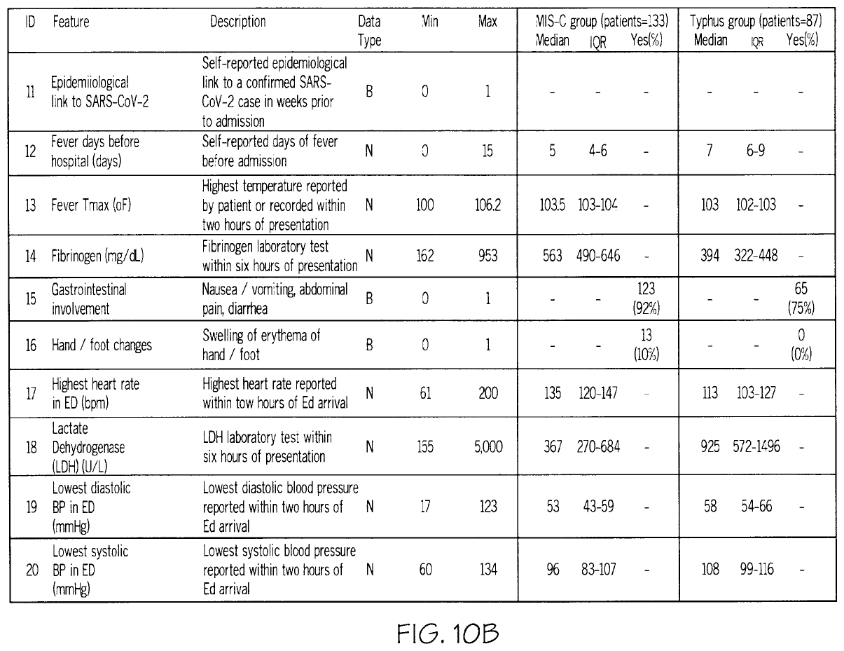

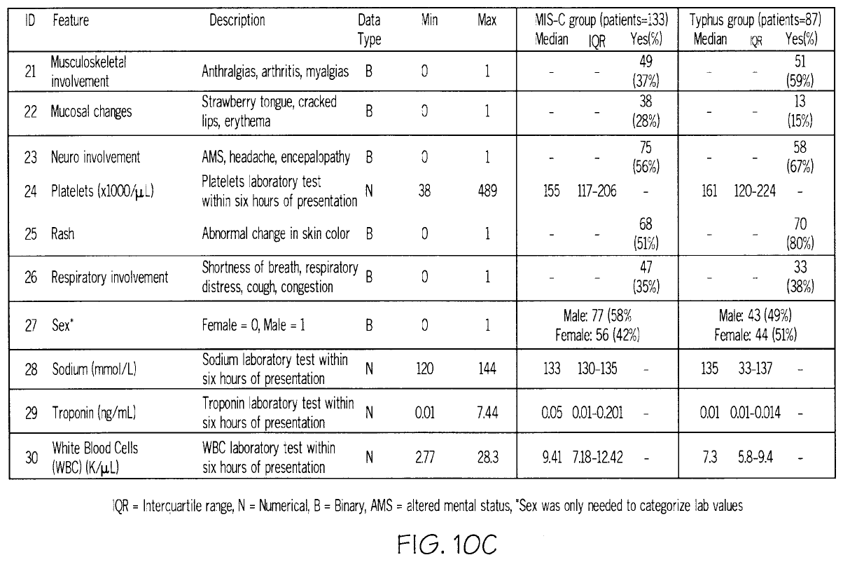

The dataset used for training and testing all models included 56 patients admitted with the first surge of MIS-C (May 15, 2020, to Nov. 30, 2020), 77 patients admitted with the delta surge of MIS-C(Sep. 1, 2021 to Oct. 31, 2021), and 87 patients admitted with murine typhus (Jan. 1, 2020, to Dec. 31, 2021) to Texas Children's Hospital and its two satellite campuses within the greater Houston area. Medical records were reviewed, with 49 demographic, clinical, and laboratory features available within six hours of the presentation obtained. Of these, 30 features were selected for this study (see Example 1.4). FIGS. 10A-10C provide a summary of the training and testing dataset.

### Example 1.10. Validation Dataset Description

The validation dataset included 111 patients with MIS-C admitted between November 2020 and June 2022. Medical records were reviewed for the 17 demographic, clinical, and laboratory features available within six hours of presentation to classify them using MET-17, where 54 of the 111 patients obtained a CI equal to or higher than that established and were classified only using MET-17. For the remaining 57, the 13 additional features were requested to be classified using MET-30.

### Example 1.11. MET-17: Fixed Points Scoring System

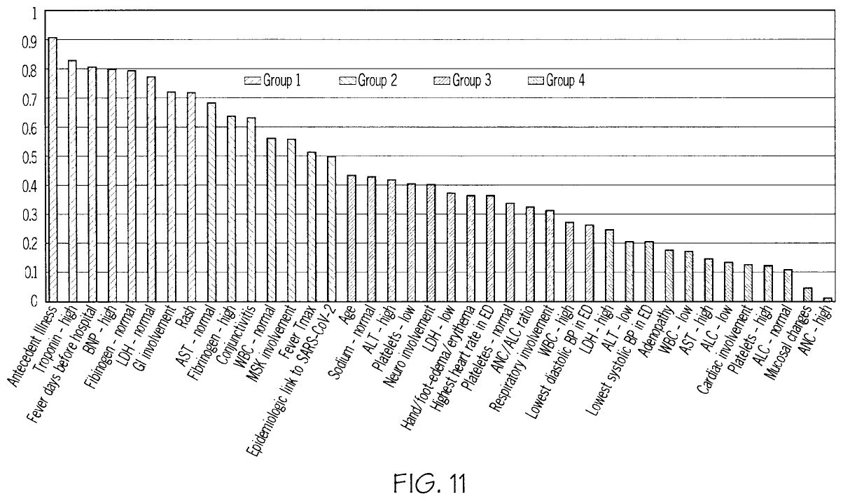

The attention values computed from MET-30 for every input are shown in FIG. 11. The group attention values for categorical inputs, calculated by the arithmetic mean of the attention value belonging to each group, are G₁=0.8, G₂=0.6, G₃=0.4, and G₄=0.1. Following the hierarchy established in Example 1.5, the constants for the continuous inputs were arbitrarily established as K₁=8, K₂=6, K₃=4, and K₄=1.

The MET-17 fixed point system for laboratory and clinical features are shown in Tables 6 and 7, respectively. The fixed point system for the continuous features can be found in Table 8. Of note, sex was not a weighted feature but was necessary to interpret normal laboratory ranges correctly, so it is the 17^{th} feature.

TABLE 6
Laboratory features for MET-17.

| Feature | If Low | If Normal | If High |
|---|---|---|---|
| ALT (U/L) | −08 | −08 | +08 |
| AST (U/L) | −03 | −11 | +11 |
| BNP (pg/mL) | +10 | +10 | −10 |
| Fibrinogen (mg/dL) | +04 | +16 | −16 |
| Sodium (mmol/L) | −02 | +02 | −02 |
| Troponin (ng/mL) | +09 | +09 | −09 |

TABLE 7
Clinical features for MET-17.

| Feature | If Positive | If Negative |
|---|---|---|
| Antecedent Illness | −02 | +02 |
| Conjunctivitis | −04 | +04 |
| Epidemiologic link to | −05 | +05 |
| SARS-CoV-2 | | |
| Rash | +03 | −03 |

TABLE 8
Continuous features for MET-17.

| Feature | Threshold | ≥ | < |
|---|---|---|---|
| Age (years) | 11 | +04 | −04 |
| ANC/ALC Ratio | 3.67 | −04 | +04 |
| Fever Before Hospital | 7 | +08 | −08 |
| (days) | | | |
| Fever Tmax (° F.) | 103 | −06 | +06 |
| Highest Heart Rate in | 124 | −04 | +04 |
| ED (BPM) | | | |

### Example 1.12. Performance Assessment

To evaluate the performance of Applicants' approach and the baseline models, Applicants computed True Positive (TP) as a correctly identified typhus patient, True Negative (TN) as a correctly identified MIS-C patient, False Positive (FP) as an MIS-C patient incorrectly identified as a typhus patient, and False Negative (FN) as a typhus patient incorrectly identified as an MIS-C patient. Applicants computed these parameters' accuracy, sensitivity, specificity, and precision.

### Example 1.13. Experimental Results

Applicants compared the performance of five statistical and machine learning models: Support Vector Machine (SVM), Dense Neural Network (DNN), Logistic Regression (LR), Random Forrest (RF), and Decision Trees (DT), to the clinical decision support system. The training, testing, and validation of all models were performed with Python 3.9.7, Tensorflow 2.2.0, Pandas 1.1.3, and Keras 2.3.0-tf running on a LINUX-based computer equipped with an AMD Ryzen 5 5600g CPU and a GeForce RTX3060 GPU.

The dataset used for training and testing was evenly divided into cohorts A and B. Information regarding the exact proportion of typhus and MIS-C patients for cohorts A, B, and validation is shown in Table 9.

TABLE 9
Training/Testing and Validation Datasets.

| Cohort | MIS-C patients | Typhus patients |
|---|---|---|
| A | 66 | 44 |
| B | 67 | 43 |
| Validation | 111 | 0 |

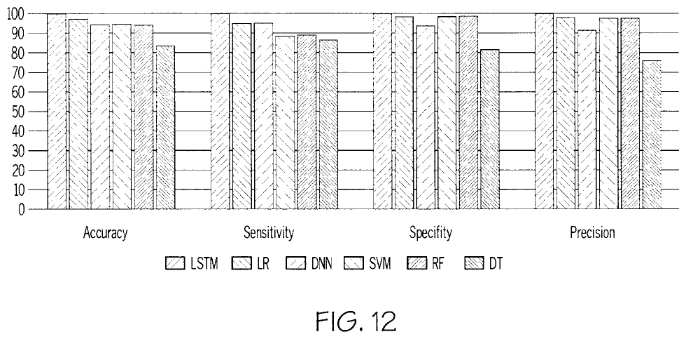

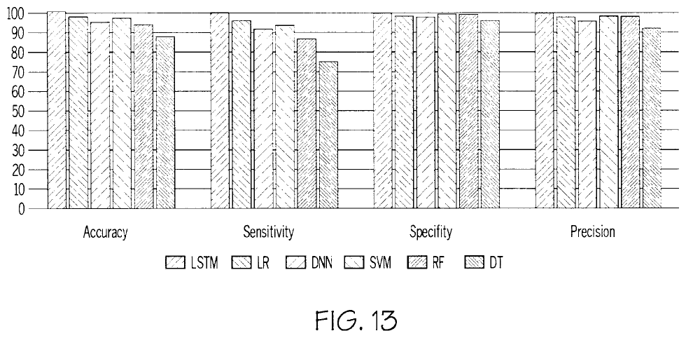

For experiment 1, all models were trained in cohort A and tested in cohort B. For experiment 2, all models were trained in cohort B and tested in cohort A. Results are shown in FIGS. 12 and 13, respectively. Finally, AI-MET was the only model assessed in the validation dataset, obtaining an accuracy of 99 percent.

### Example 1.14. Discussion

This Example uses a one-center training/testing dataset and only the MIS-C patients dataset for validation. AI-MET outperforms the baseline machine learning models used in Applicants' reported metrics. This could be partly due to the integration of the LSTM layer in MET-30, which allows learning of the contextual information generated due to the process of categorizing the laboratory features of the patients. A well-known drawback of using architectures that contain LSTM layers and the possibility of overfitting is their computational complexity. This can often lead to these architectures being computationally expensive to deploy and requiring higher resource usage. It was for this reason that MET-17 was created. AI-MET provides not only a highly effective deep learning model in the task of distinguishing patients with MIS-C from typhus patients but also a fixed scoring system that can handle approximately half of the patients (FIG. 14) by using the confidence index set at 0.95 in the experiments. In this way, AI-MET becomes a CDSS capable of being implemented in the ED without sacrificing accuracy, sensitivity, specificity, or precision.

In sum, AI-MET can be successfully employed to distinguish MIS-C from typhus based on features available within the first six hours of patient presentation. Applicants' clinical decision support system optimizes the number of clinical features needed to differentiate MIS-C from typhus with high sensitivity and specificity, and it can be implemented in the emergency department with no need for an electronic device in approximately half of the cases.

### Example 2. Distinguishing Multisystem Inflammatory Syndrome in Children from Typhus Using Artificial Intelligence: MISC Vs. Endemic Typhus (AI-MET)

In this Example, Applicants used artificial intelligence (AI) to develop a clinical decision support system that accurately distinguishes MIS-C versus Endemic Typhus (MET). Demographic, clinical, and laboratory features typically available within six hours of presentation were retrospectively extracted from the medical records of 133 patients with MIS-C and 87 patients with typhus admitted to a single quaternary system. An attention module assigned importance to inputs used to create the two-phase AI-MET. Phase 1 (MET-17) uses 17 features to arrive at a classification manually. If the pre-determined confidence level is not surpassed, 13 additional features are added to calculate MET-30 using a recurrent neural network.

While 24 of 30 features differed statistically between patients with MIS-C and typhus, the values overlapped sufficiently that the features were clinically irrelevant distinguishers as individual parameters. However, AI-MET successfully classified typhus and MIS-C with 100% accuracy. A validation cohort of 111 additional MIS-C patients with all features available was classified as MIS-C with 99% accuracy. These results further affirm that artificial intelligence can successfully distinguish MIS-C from typhus using rapidly available features. This decision support system will be a valuable tool for front-line providers facing the difficulty of diagnosing a febrile child in endemic areas.

The electronic medical record system was queried to identify patients tested for rickettsial disease by serology at Texas Children's Hospital and its two satellite campuses within the greater Houston area between Jan. 1, 2020 and Dec. 31, 2021. Acute typhus infection was defined by a positive test for Rickettsia typhi IgM antibodies. MIS-C patients were identified by rheumatology consult requests for MIS-C evaluation of febrile children at any of the three Texas Children's Hospital (TCH) campuses. All patients with MIS-C fulfilled the 2020 Centers for Disease Control and Prevention criteria for MIS-C⁵ and had positive antibodies against SARS-CoV-2 at the time of their presentation with MIS-C.

All typhus patients plus MIS-C patients from both the first surge of MIS-C (from May 15 to Nov. 30, 2020), encompassing pre-alpha and alpha variant-driven disease, and the delta variant-driven surge of MIS-C (from September 1 to Oct. 31, 2021) were included in the testing cohort used to create AI-MET (FIG. 1). Electronic medical records of these patients were reviewed for self-reported demographics, clinical presentation, and laboratory results, together totaling 49 features initially suspected to be distinguishing between MIS-C and typhus and available within six hours of presentation. If patients arrived by transfer from another hospital, first vital signs and laboratory parameters from the external institution were used. Data from an additional 160 MIS-C patients admitted to the TCH system through Jul. 31, 2022 were extracted for the 30 features needed for AI-MET and used as a validation cohort; 111 of these patients had all features needed.

### Example 2.1. Statistical Methods

Demographic, clinical, and laboratory features were compared between the MIS-C and typhus cohorts using the Mann-Whitney U test for continuous variables and Fisher's exact test for categorical variables, with p<0.05 set as the significance level.

### Example 2.2. Artificial Intelligence/Machine Learning

AI-MET is a two-phase decision support system. Phase 1 uses 17 demographic, clinical, and laboratory features to classify typhus and MIS-C manually. If the pre-assigned confidence level for classification is not surpassed using MET-17, 13 additional features are added, and, using a recurrent neural network, the patients are classified using MET-30. The process of deriving AI-MET is described in detail in Example 1, and in brief herein.

The 49 patient features collected included 21 categorical and 28 continuous variables. In addition, absolute neutrophil count (ANC) and absolute lymphocyte count (ALC) were used to generate the neutrophil to lymphocyte ratio (NLR=ANC/ALC), another continuous variable. Continuous variables were categorized as low, normal, or high using institution-specific values, leading to 73 possible model inputs generated from the 49 features. These 73 inputs were narrowed to 40 using an iterative process based on weighted values obtained through an attention module of a deep learning model during training. Through this process, the inputs with the lowest weight were removed one by one until removal impacted the testing accuracy of the model. The 40 remaining inputs were derived from 30 features (because, for example, both a high fibrinogen level and a normal fibrinogen level were weighted by attention). The 40 weighted inputs were clustered into four groups based on attention weights, and the average weight for each group was computed.

The MET-17 score is the sum of established values for a patient's categorical and continuous inputs. For categorical inputs, values were created by multiplying the average weight for the input's attention group by the difference in probability that the patient is classified as typhus or MIS-C given that the input is True or False. The obtained values were normalized, multiplied by ten, and rounded to obtain the final categorical input values, and values below one after rounding were excluded. For continuous inputs, values were created using a sign function dependent on an empirically established threshold, based on the distribution of the continuous input among patients with typhus or those with MIS-C, multiplied by a constant determined by the input's weighted attention group. Low, normal, and high inputs were compressed to simplify the system (i.e., a value that is True for high is False for both normal and low). In order to create the manual scoring system, thresholds were established at an empirically determined confidence level of 0.95, considering the distribution of the MET-17 scores of all the patients, and adjusted using a sigmoidal function. This resulted in high and low thresholds for classifying a patient as having typhus or MIS-C, respectively.

If the MET-17 score does not surpass the high confidence threshold, MET-30 is next computed, which requires a deep learning model. The deep learning model has three features: the attention module, a long short-term memory (LSTM) layer capable of learning the contextual information within the data, and a classification module composed of dense layers. Overfitting was avoided by implementing a recurrent dropout in the LSTM layer and a feed-forward dropout in the dense layers.

### Example 2.3. Cohorts

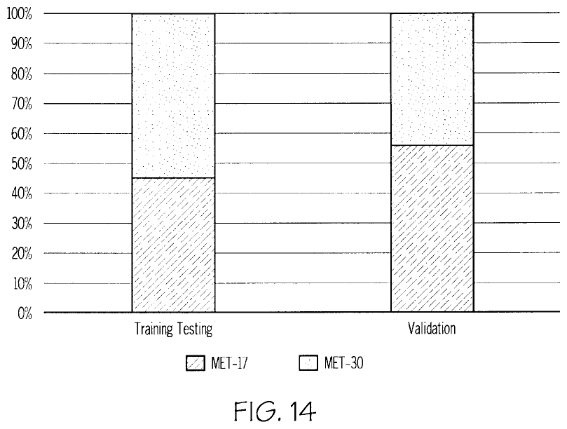

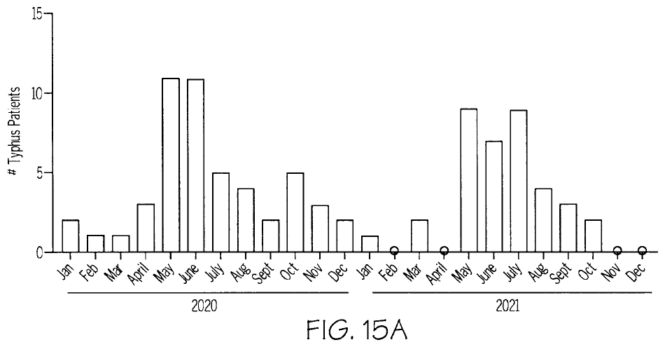

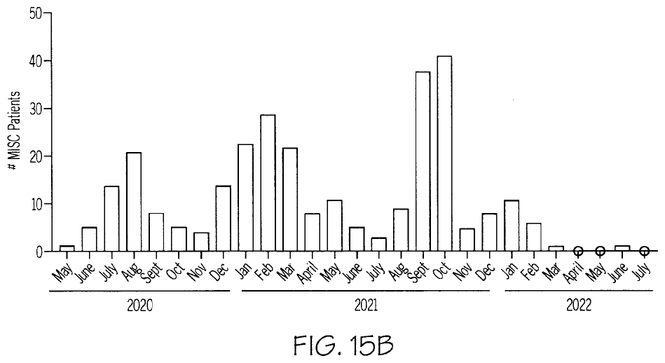

Between Jan. 1, 2020 and Dec. 31, 2021, a total of 1,268 tests for rickettsial serology were performed within three hospital systems, resulting in 159 acute typhus diagnoses (13%) as determined by the presence of positive Rickettsia typhi IgM antibodies. The 87 patients diagnosed based on testing sent during a hospital admission for an acute febrile illness (55% of those infected) formed the training typhus cohort (FIG. 15A).

From May 15, 2020 to Jul. 31, 2022, a total of 293 patients were diagnosed and treated for MIS-C during a hospitalization for an acute febrile illness within the three hospital systems. The 56 patients admitted with the first surge of MIS-C (May to November 2020) and the delta surge of MIS-C(September and October 2021) together formed the 133 patient training MIS-C cohort (FIG. 15B). The MIS-C patients used for the training cohort were selected from two different MIS-C surges driven by different SARS-CoV-2 variants to capture possible alterations in presentations across the evolution of the pandemic.

### Example 2.4. Features and Inputs

Applicants collected retrospective demographic, clinical, and laboratory data for 49 features suspected to be potentially distinguishing between typhus and MIS-C. Applicants prioritized accuracy over the number of features needed to distinguish the conditions during AI-MET development. Using the attention module of a deep learning model, Applicants narrowed to the 30 features necessary to maintain complete accuracy in distinguishing between typhus and MIS-C during training and testing.

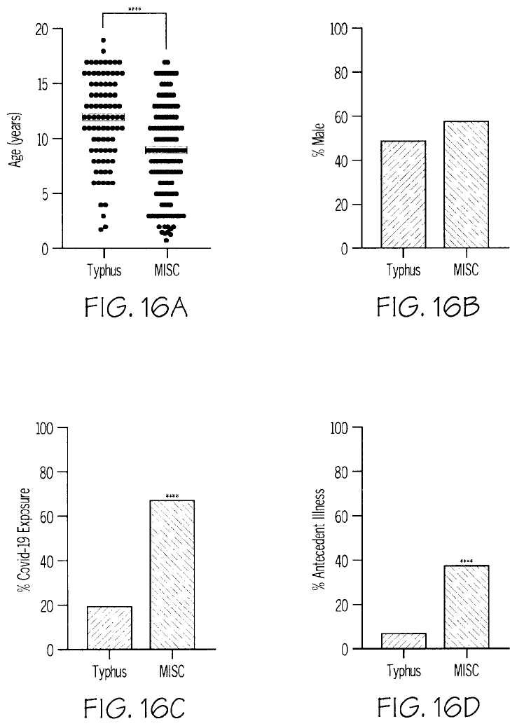

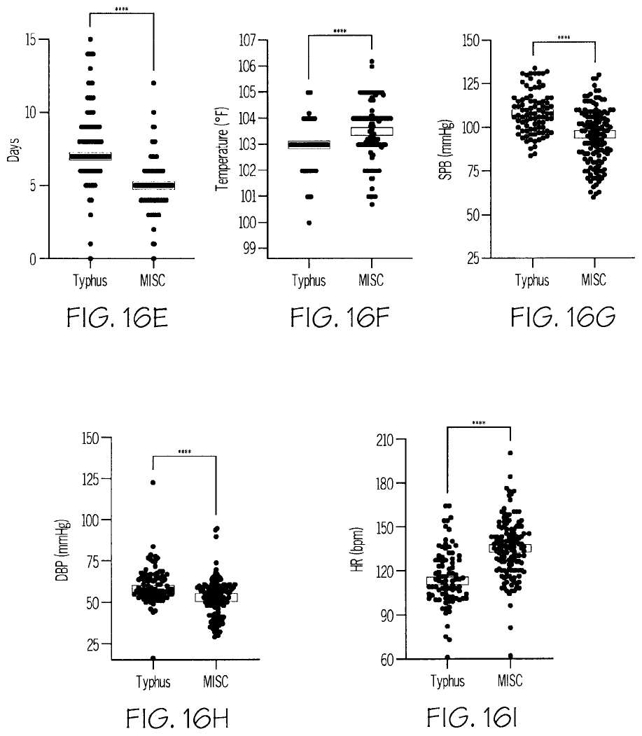

As a group, MIS-C patients were three years younger (9 [IQR 5-12] v 12 [IQR 9-15] years, p<0.0001) than those with typhus (FIG. 16A). Self-reported biologic sex did not differ between the two cohorts (FIG. 16B). Not surprisingly, patients with MIS-C were more likely to report a known close contact with a COVID-19 patient (68% v 17%, p<0.0001) in the weeks leading up to their hospitalization (FIG. 16C). MIS-C patients were also more likely (38% v 7%, p<0.0001) to report an antecedent illness leading up to their hospitalization (FIG. 16D).

MIS-C patients reported fewer days of fever (5 [IQR 4-6]v 7 [IQR 6-9] days, p<0.0001) before hospital presentation (FIG. 16E) but higher maximum temperature (39.7 [IQR 39.4-40]v 39.4 [IQR 38.9-39.4]° Celsius, p<0.0001) than typhus patients (FIG. 16F). The median lowest systolic blood pressures (96 [IQR 83-107]v 108 [IQR 99-116] mmHg, p<0.0001), lowest diastolic blood pressures (53 [IQR 43-59]v 58 [IQR 54-66] mmHg, p<0.0001), and highest heart rates (135 [IQR 120-147]v 113 [IQR 103-127] beats/minute, p<0.0001) recorded in the first two hours of presentation were found in MIS-C patients (FIGS. 16G-I), suggestive of more severe illness.

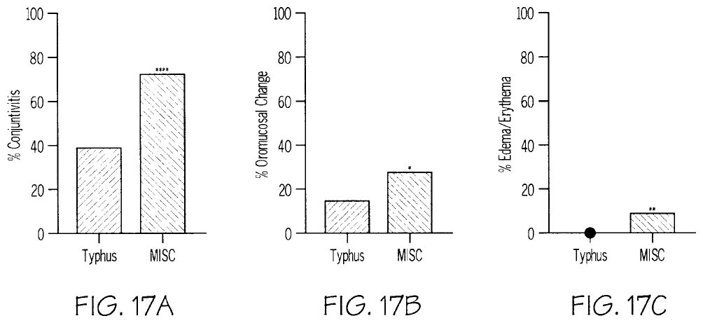

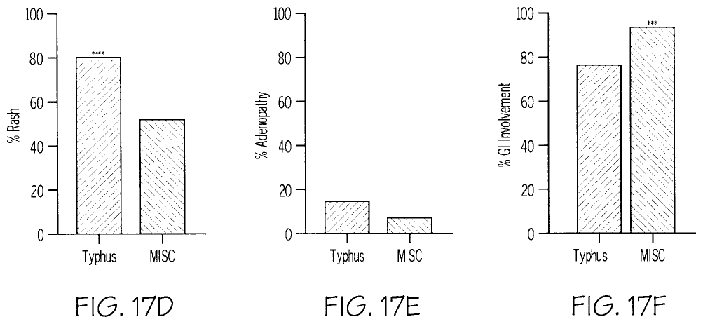

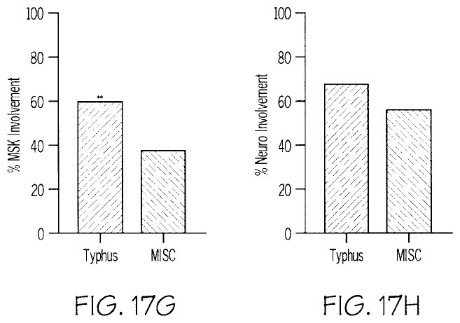

Clinically, MIS-C patients were more likely to have conjunctivitis (72% v 39%, p<0.0001), oromucosal changes (28% v 15%, p=0.028), and hand or foot edema or erythema (10% v 0%, p<0.01), while typhus patients were more likely to have a rash (80% v 51%, p<0.0001) than patients with MIS-C (FIGS. 17A-D). The presence of cervical adenopathy did not differ (FIG. 17E). Patients with MIS-C reported more gastrointestinal symptoms (FIG. 17F) of abdominal pain, vomiting, and diarrhea (92% v 75%, p<0.001) and typhus patients reported more arthralgias and myalgias (musculoskeletal symptoms, 59% v 37%, p<0.01, FIG. 17G). Neurologic symptoms, including headache, respiratory symptoms (shortness of breath, cough, congestion), and cardiac symptoms (chest pain, palpitations) did not differ (FIGS. 17H-J).

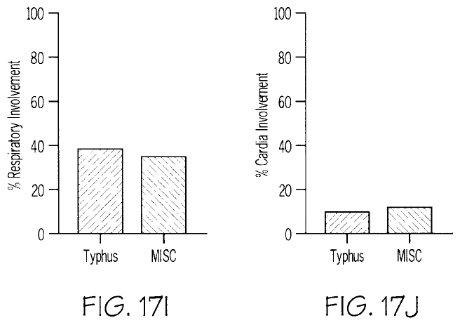

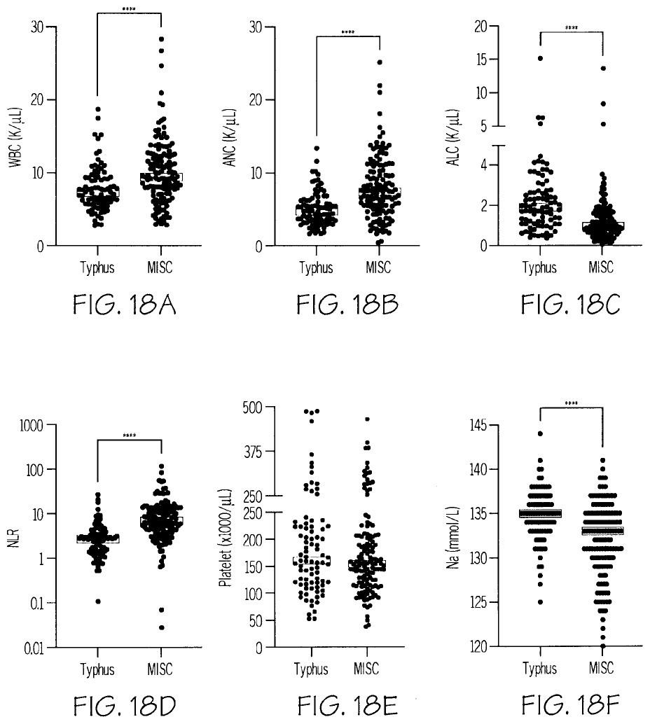

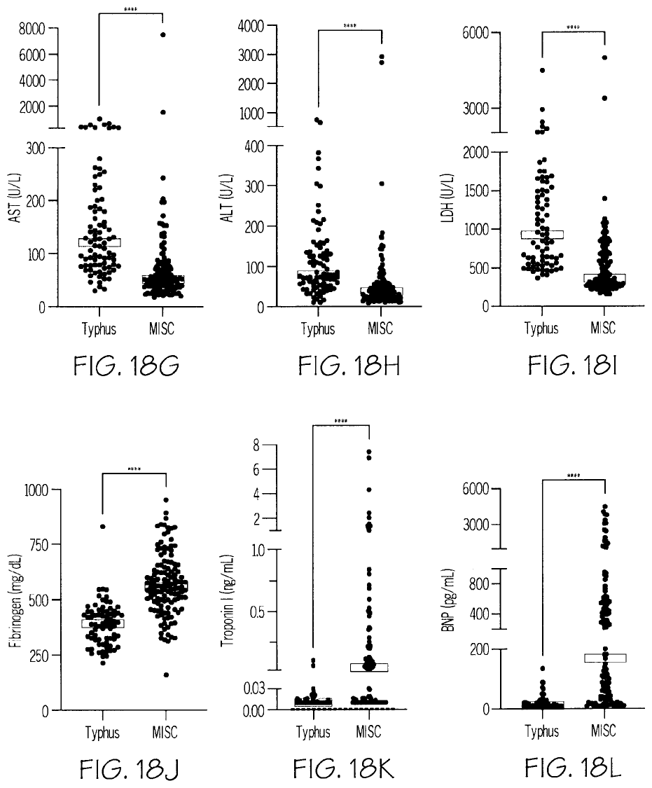

Except for platelet count, hematologic laboratory parameters were highly significantly different (p<0.0001), including higher white blood cell count (WBC, 9.4 [IQR 7-2-12.4]v 7.3 [IQR 5.8-9.4]K/μL), absolute neutrophil count (ANC, 7.4 [IQR 5-10.3]v 4-7 [IQR 3.4-6.2]K/μL) and neutrophil to lymphocyte ratio (NLR, 7 [IQR 4-13.4]v 2.8 [IQR 1.4-4]) and lower absolute lymphocyte count (ALC, 0-96 [IQR 0.66-1.7]v 1-9 [IQR 1.1-2.7]K/μL) in MIS-C patients (FIGS. 18A-E). MIS-C patients were more likely to have hyponatremia with median sodium (Na) of 133 [IQR 130-135]v 135 [IQR 133-137], but had lower aspartate aminotransferase (AST, 52 [IQR 38-77]v 121 [IQR 78-192]U/L), alanine aminotransferase (ALT, 37 [IQR 22-56]v 80 [IQR 53-137]U/L), and lactate dehydrogenase levels (LDH, 367 [IQR 270-684]v 925 [IQR 572-1496]U/L), all p<0.0001 (FIGS. 18F-I). Fibrinogen was significantly higher in MIS-C patients (median 563 [IQR 490-646]v 394 [IQR 322-448] mg/dL, p<0-0001, FIG. 18J); other inflammatory markers (ferritin, C-reactive protein, erythrocyte sedimentation rate, procalcitonin, and D-dimer) were not weighted for attention.

Cardiac biomarkers were the most likely features to be discriminatory on their own, but still overlapped. A majority of MIS-C patients did have elevated troponin I and nearly all typhus patients had a normal troponin I. Still, there were two typhus patients with elevated values and 45% of MIS-C patients (58/129 with data available) had normal troponin I (median troponin I for MIS-C 0.05 [IQR 0.01-0.2]v typhus 0.01 [IQR 0.01-0.014] ng/mL, p<0-0001, FIG. 18K). None of the typhus patients had an elevated B-type natriuretic peptide (BNP). Still, the median BNP value for MIS-C patients was also within the range of normal (median for MIS-C 167 [IQR 41-560]v typhus 12 [IQR 10-26] pg/mL, p<0.0001, FIG. 18L).

### Example 2.5. AI-MET

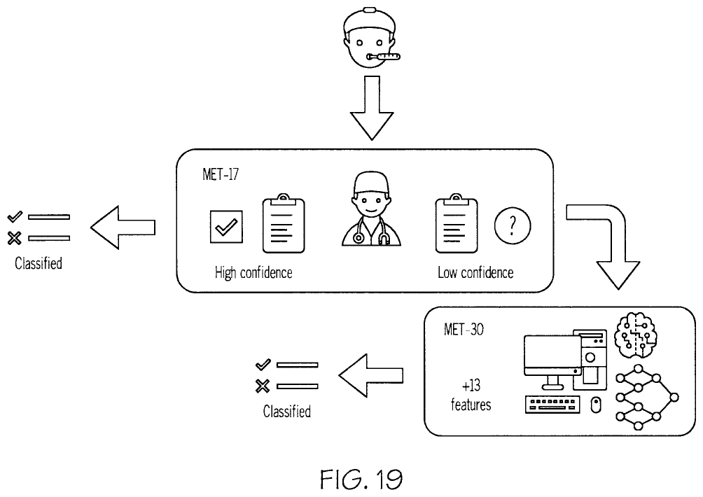

As set forth in Example 1, AI-MET was generated using a deep learning model. While developing AI-MET, Applicants prioritized the desire for a scoring system that could be used manually, without a computer or software, to arrive at a classification, as has previously been accomplished for other clinical questions. To maintain Applicants' other priority, accuracy, this resulted in generating a two-phase decision support system (FIG. 19) to distinguish MIS-C from typhus, including an initial manual step. Phase 1 uses 17 of the 30 weighted features (Table 10), resulting in 15-point quantities summed for a total score. Of note, sex was not a weighted feature but must be collected and used to interpret AST, ALT, and LDH per institutional norms. Further, ANC and ALC were not individually weighted but used together to calculate the NLR. If the total score for a patient is 62 points or above or (−)35 points or below, there is appropriate confidence that the patient has been classified correctly as either typhus or MIS-C, respectively. For the 87 typhus and 133 MIS-C patients from the training cohort, MET-17 accurately classified 40 (46%) typhus and 60 (45%) MIS-C patients.

Suppose sufficient points are not tallied to surpass the confidence level for classification using MET-17. In that case, the 13 remaining weighted features must also be used to calculate the MET-30 score using a recurrent neural network. All typhus and MIS-C patients in the training cohort that could not be classified with confidence using MET-17 were correctly classified using MET-30, as it was designed for complete accuracy.

### Example 2.6. Internal Validation

An additional 160 patients with MIS-C were diagnosed and treated within a hospital system during the included time period (FIG. 15B). These MIS-C patients were considered to form a validation cohort to challenge the AI-MET decision support system. The features needed to perform AI-MET were available for 111 of these MIS-C patients. At the initial MET-17 phase, 62 out of the 111 patients (56%) were classified with sufficient confidence. MET-30 was then performed for the remaining 49 individuals, correctly classifying 48/49 MIS-C patients (98%). Therefore, AI-MET achieved 99% accuracy (110/111 MIS-C patients).

### Example 2.7. Discussion

The presentations of febrile patients with MIS-C mimic a number of other common childhood conditions, including Kawasaki Disease and typhus. Consequently, as COVID-19 moves towards endemicity while the incidence of MIS-C becomes sporadic, pediatric providers must maintain a high level of clinical suspicion for MIS-C when faced with a febrile child in endemic areas. Though there were statistically significant differences in demographic, clinical, and laboratory features between the patients with MIS-C and typhus, these differences were sufficiently overlapping that they could not be used to confidently distinguish between patients if used alone. Instead, using machine learning, Applicants developed a two-phase clinical decision support system (FIG. 19) to help rapidly and accurately classify patients as MIS-C versus typhus. The ability to use AI-MET with features typically available within hours of presentation will be able to guide providers with management, which varies greatly between these conditions.

TABLE 10
MET-17 Features and Scoring.

| Features with Thresholds | Threshold | Points if ≥ | Points if < |
|---|---|---|---|
| Age (years) | 11 | (+)4 | (−)4 |
| Fever Before Presentation (days) | 7 | (+)8 | (−)8 |
| Tmax (° C.) | 39.4 (103° F.) | (−)6 | (+)6 |
| Highest Heart Rate (bpm) | 124 | (−)4 | (+)4 |
| NLR (ANC/ALC) | 3.67 | (−)4 | (+)4 |
| Features with Multiple Inputs | Points if Low | Points if WNL | Points if High |
| AST (U/L) | (−)3 | (−)11 | (+)11 |
| ALT (U/L) | (−)8 | (−)8 | (+)8 |
| Sodium (mmol/L) | (−)2 | (+)2 | (−)2 |
| Troponin I (ng/ml) | (+)9 | (+)9 | (−)9 |
| BNP (pg/mL) | (+)10 | (+)10 | (−)10 |
| Fibrinogen (mg/dL) | (+)4 | (+)16 | (−)16 |
| Categorical Features | | Points if Yes | Points if No |

| | | |
|---|---|---|
| Epidemiologic Link to COVID-19 | (−)5 | (+)5 |
| Antecedent Illness | (−)2 | (+)2 |
| Conjunctivitis | (−)4 | (+)4 |
| Rash | (+)3 | (−)3 |

The patient is classified as typhus if the total score is ≥62.
The patient is classified as MIS-C if the total score is ≤(−)35.
If the total score falls between (−)35 and 62, proceed to MET-30.
ALC absolute lymphocyte count; ALT, alanine aminotransferase, ANC, absolute neutrophil count; AST, aspartate aminotransferase; BNP, B-type natriuretic peptide; bpm, beats per minute; ° C., degree Celsius; ° F., degree Fahrenheit; NLR, neutrophil to lymphocyte ratio, Tmax, maximum temperature; WNL, within normal limits.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above but is only limited by the claims, including all equivalents of the subject matter of the claims. The disclosures of all patents, patent applications, and publications cited herein are hereby incorporated herein by reference to the extent that they provide procedural or other details consistent with and supplementary to those set forth herein.

What is claimed is:

1. A method of distinguishing between a first and a second condition in a subject, said method comprising:
receiving a first set of health-related data from the subject;
determining if a confidence index crosses a certain threshold; and
distinguishing between the first and the second condition in the subject from the first set of health-related data if the confidence index crosses the threshold, or
receiving a second set of health-related data from the subject if the confidence index does not cross the threshold and distinguishing between the first and the second condition in the subject from the first set and the second set of health-related data.

2. The method of claim 1, wherein at least one of the first set and the second set of health-related data comprise laboratory test results, wherein the laboratory test results are selected from the group consisting of white blood cell (WBC) count, absolute lymphocyte count (ALC), absolute neutrophil count (ANC), platelet count, sodium count, troponin count, B-type natriuretic peptide (BNP) count, fibrinogen count, D-dimer count, aspartate aminotransaminase count (AST), lactate dehydrogenase count (LDH), alanine aminotransaminase count (ALT), ANC/ALC ratio, ferritin count, and combinations thereof.

3. The method of claim 1, wherein at least one of the first set and the second set of health-related data comprise clinical data, wherein the clinical data are selected from the group consisting of antecedent illness; conjunctivitis; epidemiologic link to COVID-19; oromucosal changes; hand or foot edema or erythema; rash; cervical adenopathy; nausea, vomiting, pain, diarrhea, or other gastrointestinal involvements; arthralgias, myalgias, or other musculoskeletal involvements; altered mental status (AMS), seizure, paresthesias, headache, or other neurological involvements; shortness of breath, cough, congestion, or other respiratory involvements; chest pain, palpitations or other cardiac involvements; and combinations thereof.

4. The method of claim 1, wherein at least one of the first set and the second set of health-related data comprise vital signs, wherein the vital signs are selected from the group consisting of fever, heart rate, blood pressure, and combinations thereof.

5. The method of claim 1, wherein at least one of the first set and the second set of health-related data comprise demographic data, wherein the demographic data are selected from the group consisting of age, gender, and combinations thereof.

6. The method of claim 1, wherein the first set and the second set of health-related data are each independently selected from the group consisting of age; gender; epidemiologic link to COVID-19; antecedent illness; fever; maximum reported or recorded temperature during the first six hours of the subject's visit to a clinic or hospital; lowest systolic blood pressure during the first six hours of the subject's visit to a clinic or hospital; lowest diastolic blood pressure during the first six hours of the subject's visit to a clinic or hospital; highest heart rate during the first six hours of the subject's visit to a clinic or hospital; conjunctivitis; oromucosal changes; hand or foot edema or erythema; rash; cervical adenopathy; nausea, vomiting, pain, diarrhea, or other gastrointestinal involvements; arthralgias, myalgias, or other musculoskeletal involvements; altered mental status (AMS), seizure, paresthesias, headache, or other neurological involvements; shortness of breath, cough, congestion, or other respiratory involvements; chest pain, palpitations or other cardiac involvements; white blood cell (WBC) count; absolute neutrophil count (ANC); absolute lymphocyte count (ALC); platelet count; sodium count; aspartate aminotransferase (AST) count, alanine aminotransferase (ALT) count; lactate dehydrogenase (LDH) count; fibrinogen count; troponin count; B-type natriuretic peptide (BNP) count; ANC/ALC ratio; and combinations thereof.

7. The method of claim 1, wherein the first set and the second set of health-related data are obtained during the first six hours of the subject's visit to a clinic or hospital.

8. The method of claim 1, wherein at least the steps of receiving the first set of health-related data from the subject; and determining if the confidence index crosses a certain threshold occur without the use of a computing device.

9. The method of claim 1, wherein at least the steps of receiving the second set of health-related data from the subject if the confidence index does not cross the threshold; and distinguishing between the first and the second condition in the subject from the first set and the second set of health-related data occurs through the utilization of a computing device.

10. The method of claim 9, wherein the computing device comprises a machine-learning algorithm trained on the first and second sets of health-related data, wherein the receiving comprises feeding the health-related data to the machine-learning algorithm, and wherein the distinguishing comprises generating an output from the machine-learning algorithm.

11. The method of claim 10, wherein the machine learning algorithm comprises:
an attention module, wherein the attention module is operable to receive health-related data as inputs and provide values that weigh the inputs based on relevance;
a long short-term memory (LSTM) layer operable to receive the weighted inputs from the attention module and learn contextual information within the inputs; and
a classification module operable to receive the inputs from the LSTM layer, wherein the classification module comprises a plurality of connected dense layers, wherein the dense layers are operable to distinguish between the first and the second condition and generate a corresponding output.

12. The method of claim 1, wherein each of the first condition and second condition is independently selected from the group consisting of Multisystem Inflammatory Syndrome in Children (MIS-C), Kawasaki Disease, a febrile illness, typhus, endemic typhus, or toxic shock syndrome (TSS).

13. The method of claim 1, wherein the first condition comprises Multisystem Inflammatory Syndrome in Children (MIS-C) and the second condition comprises endemic typhus.

14. The method of claim 1, wherein the first condition comprises Multisystem Inflammatory Syndrome in Children (MIS-C) and the second condition comprises Kawasaki Disease.

15. The method of claim 1, wherein the subject is a human being.

16. The method of claim 1, further comprising a step of making a treatment decision based on distinguishing between the first and the second condition in the subject.

17. The method of claim 16, wherein the treatment decision comprises monitoring the course of the condition.

18. The method of claim 16, wherein the treatment decision comprises administering a therapeutic agent to the subject.

19. The method of claim 18, wherein the therapeutic agent is selected from the group consisting of anti-viral drugs, anti-inflammatories, steroids, antibiotics, and combinations thereof.

20. A clinical decision support system for distinguishing between a first and a second condition in a subject, said system comprising:
instructions for receiving a first set of health-related data from the subject;
instructions for determining if a confidence index crosses a certain threshold; and
instructions for distinguishing between the first and the second condition in the subject from the first set of health-related data if the confidence index crosses the threshold, or
instructions for receiving a second set of health-related data from the subject if the confidence index does not cross the threshold and distinguishing between the first and the second condition in the subject from the first set and the second set of health-related data.

21-38. (canceled)

39. A computer-implemented method of distinguishing between a first and a second condition in a subject, said method comprising:
receiving a plurality of health-related data from the subject, wherein the receiving comprises feeding the health-related data to a machine-learning algorithm trained on the plurality of health-related data; and
distinguishing between the first and the second condition in the subject from the plurality of health-related data, wherein the distinguishing comprises generating an output from the machine-learning algorithm.

40-54. (canceled)

55. A computing device operable to distinguish between the first and the second condition in a subject, said computing device comprising:
a machine-learning algorithm trained on a plurality of health-related data;
programming instructions for receiving a plurality of health-related data from the subject and feeding the health-related data into the machine-learning algorithm; and
programming instructions for generating an output from the machine-learning algorithm that distinguishes between the first and the second condition in the subject from the plurality of health-related data.

56-70. (canceled)
